(19) United States
(12) Patent Application Publication
(10) Pub. No.: US 2026/0262762 A1
(43) Pub. Date: Sep. 10, 2026

(54) Apparatuses and Materials for Enhancing the Effects of Exercise

(71) Applicant: Michael Rudan, Ontario (CA)

(72) Inventor: Michael Rudan, Ontario (CA)

(21) Appl. No.: 19/167,495

(22) PCT Filed: Feb. 29, 2024
(86) PCT No.: PCT/CA2024/050252

§ 371 Date: Sep. 22, 2025

Related U.S. Application Data
(60) Provisional application No. 63/453,895, filed on Mar. 22, 2023.

(51) Int. Cl. A41D 13/00; A41D 1/089; A61F 5/03

(52) U.S. Cl. CPC A41D 13/0015; A41D 1/089; A61F 5/03; A41D 2300/322; A41D 2300/326

(57) ABSTRACT

Apparatuses are discussed comprising materials for enhancing physical exercise, in particular, compressible materials that can be incorporated into apparatuses such as athletic footwear, apparel, fitness equipment and the like to provide a proprioceptive effect on a person when using the apparatuses during physical exercise. The compressible materials can be made of one or more of rubber, thermoplastic rubber, foam rubber, polyurethane rubber, silicone rubber, thermoplastic resin and thermoplastic elastomer. Positioning the compressible material such that it is between the user and any surface for example when exercising, requires the person to maintain balance while performing the exercise by using other muscles that would not ordinarily be used during the exercise, and provides proprioceptive feedback to the person's central nervous system.

### CROSS-REFERENCE TO RELATED APPLICATIONS:

This application claims priority of United States provisional patent application Ser. No. 63/453,895 filed Mar. 22, 2023, which is incorporated by reference into this application in its entirety.

### TECHNICAL FIELD:

The present disclosure is related to the field of apparatuses comprising materials for enhancing physical exercise, in particular, compressible materials that can be incorporated into apparatuses such as athletic footwear, apparel, fitness equipment and the like to provide a proprioceptive effect on a person when using the apparatuses during physical exercise.

BACKGROUND:

It is often difficult for people who are not in the best of physical shape to improve their physical well-being and health. Also, those who were more physically active and fit when they were younger tend to become more sedentary as they age, and their physical well-being and health can decline. In addition, those who have suffered a physical injury, notwithstanding that they may have been physically fit and healthy prior to the injury, may find it difficult to continue exercising as they could prior to their injury. In all of these cases, there is a need for improved physical fitness footwear, apparel and equipment that can enable anyone to enhance their exercise regime by causing them to use additional muscles to maintain their balance while exercise while lowering the amount of weight or force required so as to not overtax the person's joints and muscles and improve proprioception, especially if they had been previously sedentary or are recovering from an injury.

In The Role of Proprioception in the Management and Rehabilitation of Athletic Injuries (SM Lephart et al., The American Journal of Sports Medicine, Vol. 25, No. 1) proprioception is defined as a specialized sensory modality of touch and encompasses the sensations of joint movement and joint position. Proprioception contributes to motor programing for neuromuscular control required for precision movements and also contributes to muscle reflexes, providing dynamic joint stability. Proprioceptive receptors found in the skin, muscles and joints provide input to the central nervous system (“CNS”) and, along with visual and vestibular centres, contributes afferent information to the CNS regarding body position and balance. A sports injury or other musculoskeletal trauma can lead to proprioceptive defects. Proprioceptive rehabilitation seeks to retrain altered afferent pathways to enhance sensation of joint movement. There is a need for improved physical fitness footwear, apparel and equipment that can enable anyone to train or retrain afferent pathways to improve functional stability of the person's joints and body.

It is, therefore, desirable to provide apparatuses comprising compressible materials that can be incorporated therein to provide a proprioceptive effect and, thus, enhance the effects of physical exercise.

### SUMMARY:

Apparatuses comprising compressible material, such as athletic footwear, apparel, fitness equipment and the like, is provided to provide a proprioceptive effect to enhance the effects of physical exercise. In some embodiments, the apparatuses can comprise the materials and devices as described in international patent application no. PCT/CA2017/050874, which is incorporated by reference into this application in its entirety.

In some embodiments, compressible protrusions can be added to the anterior (palm) or dorsal (top) region as well as on the phalange region of gloves used for exercising, weight lifting or running, to the soles, toes or instep of shoes used for exercising, weight lifting or running, to the soles of galoshes that can be slipped onto regular shoes, to the soles of insoles for shoes, to the bottom of exercise mats, to the equipment side of barbell wraps, to the outside of joint wraps for use with elbows or knees, athletic apparel such as pants, tights, leggings, shirts, hoodies or one-piece exercise clothing, to footwear such as socks, shoes, or shoe covers, and to fitness or gym flooring. The addition of the compressible protrusions can operate similarly to exercise equipment that comprise compressible spheres or hemispheres, such as used in Pilates and in other forms of exercising. The compressible hemispheres can also cause the user to actively hold the muscles, joints and body in a proper alignment providing proprioceptive feedback to the CNS. These voluntary movements can be repeated and stored as central commands. This patterning of the brain allows various skills to be performed without continuous without continuous reference to consciousness and can increase the conscious and unconscious awareness of the joint or limb in motion.

The placement of compressible protrusions on the bottom of weight lifting shoes on a person lifting weights, as an example, requires the person hold the body in proper alignment, adding proprioceptive elements to the exercise to assist in proper patterning of the CNS and to exert additional effort and use other muscles, such as their core muscles, to maintain balance in addition to the weight lifting exercise they are carrying out, such as bicep curls, front and side dumbbell lateral raises and the like. In such shoes, the sole can have one or more different sized compressible hemispheres, such as larger diameter hemispheres under the ball or heel of the shoe, and smaller diameter hemispheres disposed between and around the larger hemispheres. In some embodiments, a running shoe can comprise one or more compressible hemispheres disposed on the sole, facing downwards. In some embodiments, compressible hemispheres can be placed on the bottom of galoshes, which can be slipped onto regular shoes. In other embodiments, an insole having compressible hemispheres on the bottom thereof, facing downwards, can be used in regular shoes to similar effect.

In some embodiments, exercise equipment can comprise such compressible hemispheres. In one example, bar wraps can have one or more such compressible hemispheres disposed on one side of the wrap, the side that faces outwardly towards the user such that the user contacts the compressible hemispheres directly when grabbing the bar. In other embodiments, a workout mat or floor covering can comprise one or more compressible hemispheres extending upwards from the floor when the mat or floor covering is laid out for use on the floor. In some embodiments, a joint wrap for elbows or knees can comprise one or more compressible hemispheres extending outwardly from the wrap. The joint wrap can be comprised of an elastic tube that can stretch and slip onto a person's arm or leg. The placement of the compressible hemispheres can enable the user to perform exercises where contact by the elbows or knees to a hard surface, such as a floor or wall, via the compressible hemispheres.

In some embodiments, athletic apparel such as pants, tights, leggings, shirts, hoodies or one-piece exercise clothing can comprise one or more compressible hemispheres extending outwardly from the apparel. The compressible hemispheres can be affixed to the apparel. In some embodiments the compressible hemispheres can be removably attached to the apparel. In some embodiments the apparel can comprise pockets for removably attaching the compressible hemispheres at the desired locations. In other embodiments the compressible hemispheres can be attached using Velcro®, hooks, buttons, snaps, straps or other suitable removable attachment as well known to those skilled in the art. The placement of the compressible hemispheres can enable the user to perform exercises where there can be contact by the elbows, knees or other body part to a hard surface, such as a floor or wall, via the compressible hemispheres.

In some embodiments, a sock, shoe cover, or other foot covering can comprise one or more compressible hemispheres disposed on the sole, the toe portion, the heal portion, the leg portion or elsewhere on the foot covering, protruding outward.

In some embodiments, compressible material can be positioned so as to be between the toes. The compressible material between the toes can activate the nerves in the toes and provide proprioceptive feedback to the CNS.

In some embodiments, the compressible hemispheres can be comprised of an elastomeric material, as well known to those skilled in the art. In some embodiments, the elastomeric material can comprise can comprise one or more of a group comprising of rubber, thermoplastic rubber, foam rubber, polyurethane rubber, silicone rubber, thermoplastic resin and thermoplastic elastomer. In further embodiments, the exterior of the compressible hemisphere can be composed of a durable material, such as plastic or synthetic fiber and the interior of the compressible protrusion can comprise one or more of a liquid, a gel and a gas, such as air or combinations thereof.

Broadly stated, in some embodiments, an improved workout mat can be provided for enhancing the effects of physical exercise for a person, the mat comprising an upward-facing surface and a downward-facing surface, the improvement comprising a plurality of compressible protrusions disposed on the upward-facing surface, extending upwardly therefrom, the plurality of compressible protrusions spaced apart from each in a substantially uniform configuration.

Broadly stated, in some embodiments, each of the plurality of compressible protrusions can be substantially hexagonal in configuration.

Broadly stated, in some embodiments, the plurality of compressible protrusion can be disposed in a predetermined pattern on the upward-facing surface.

Broadly stated, in some embodiments, the predetermined pattern can comprise an upper location and a lower location disposed on the improved workout mat.

Broadly stated, in some embodiments, the predetermined pattern can further comprise a middle location disposed between the upper and lower locations.

Broadly stated, in some embodiments, the improved workout mat can further comprise interlocking members disposed along at least one edge thereof, wherein a plurality of the improved workout mat is configured as a floor covering.

Broadly stated, in some embodiments, the upward-facing surface can comprise at least one fastening strip of a hook and loop fastening mechanism, and wherein at least some of the plurality of compressible protrusions can be disposed on a pad configured for attaching to the at least one fastening strip Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, a spinal rocker can be provided comprising: a planar member comprising an upward-facing surface and a downward-facing surface; a rib member disposed on the downward-facing surface; and a plurality of compressible protrusions disposed on the upward-facing surface, the plurality of compressible protrusions spaced apart from each in a substantially uniform configuration.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, a spinal mat can be provided, comprising: at least one spine support block; a lower body support block; and a plurality of compressible protrusions extending outwardly from the at least one spine support block and the lower body support block, the plurality of compressible protrusions spaced apart from each in a substantially uniform configuration.

Broadly stated, in some embodiments, each of the plurality of compressible protrusions can be substantially hexagonal in configuration.

Broadly stated, in some embodiments, wherein one or both of the at least one spine support block and the lower support block can be comprised of compressible thermoplastic.

Broadly stated, in some embodiments, wherein the compressible thermoplastic can comprise closed cell ethylene-vinyl acetate copolymer foam.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, a footwear wrap for a shoe can be provided, comprising: a harness configured to place onto the shoe; and a sole member operatively coupled to the harness, the sole member comprising a plurality of compressible protrusions extending upwardly therefrom and configured to contact the shoe when the harness is placed on the shoe, the plurality of compressible protrusions spaced apart from each in a substantially uniform configuration.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, an abdominal exercise belt can be provided for placing around an abdomen of a person, comprising: a belt member comprising first and second ends, the belt member further comprising an outward-facing surface and an inward-facing surface, the inward-facing surface configured to contact the abdomen of the person when the belt is placed therearound; a plurality of compressible protrusions extending from the inward-facing surface; and a fastening mechanism configured for securing the abdominal exercise belt to the person.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, the fastening mechanism can comprise a hook and loop fastener.

Broadly stated, in some embodiments, the abdominal exercise can comprise an inflatable bladder disposed therein.

Broadly stated, in some embodiments, an arm wrap can be provided for placing around an arm of a person, comprising: a band member comprising first and second ends, the band member further comprising an outward-facing surface and an inward-facing surface, the inward-facing surface configured to contact the arm of the person when the band is placed therearound; a plurality of compressible protrusions extending from the inward-facing surface; and a fastening mechanism configured for securing the arm wrap band to the person.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, a leg wrap band can be provided for placing around a leg of a person, comprising: a band member comprising first and second ends, the band member further comprising an outward-facing surface and an inward-facing surface, the inward-facing surface configured to contact the leg of the person when the band is placed therearound; a plurality of compressible protrusions extending from the inward-facing surface; and a fastening mechanism configured for securing the leg wrap band to the person.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, the fastening mechanism can comprise a hook and loop fastener.

Broadly stated, in some embodiments, the leg wrap band can comprise an inflatable bladder disposed therein.

Broadly stated, in some embodiments, a garment can be provided for being worn by a person during exercise, comprising: a pair of shorts configured to be worn by the person about their waist, hips and thighs, the pair of shorts comprising an outer shell; an inner liner disposed within the outer shell, the inner liner comprising a plurality of compressible protrusions extending inwardly therefrom towards the person when the pair of shorts are worn by the person; and a fastening mechanism configured to secure the pair of shorts to the person.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, the fastening mechanism can comprise one or more of a fly zipper, a waistband fastener, an outer seam zipper and an inner seam zipper.

Broadly stated, in some embodiments, the garment can further comprise at least one inflatable bladder disposed therein.

Broadly stated, in some embodiments, a garment can be provided for being worn by a person during exercise, comprising: a shirt configured to be worn by the person on their torso, the shirt comprising an outer shell; an inner liner disposed within the outer shell, the inner liner comprising a plurality of compressible protrusions extending inwardly therefrom towards the person when the shirt is worn by the person; and a fastening mechanism configured to secure the shirt to the person.

Broadly stated, in some embodiments, each of the plurality of compressible protrusions can be substantially hexagonal in configuration.

Broadly stated, in some embodiments, each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, the fastening mechanism can comprise one or more of a zipper, one or more buttons, one or more clasps and one or more snap fasteners.

Broadly stated, in some embodiments, the garment can further comprise at least one inflatable bladder disposed therein.

Broadly stated, in some embodiments, a step up bench can be provided, comprising: a seat portion; a back rest; and a plurality of compressible protrusions extending outwardly from one or both of the seat portion and the back rest, the plurality of compressible protrusions spaced apart from each in a substantially uniform configuration.

Broadly stated, in some embodiments, each of the plurality of compressible protrusions disposed on the step up bench can be substantially hexagonal in configuration.

Broadly stated, in some embodiments, each of the plurality of compressible protrusions disposed on the step up bench can comprise one or more of a group comprising rubber, thermoplastic rubber, foam rubber, polyurethane rubber, silicone rubber, thermoplastic resin and thermoplastic elastomer.

Broadly stated, in some embodiments, a wrap can be provided for a dumbbell handle or a bar for use with exercise equipment, comprising: a substrate configured to wrap around the dumbbell handle or the bar; and a plurality of compressible protrusions extending outwardly from the substrate, the plurality of compressible protrusions spaced apart from each in a substantially uniform configuration.

Broadly stated, in some embodiments, each of the plurality of compressible protrusions disposed on the wrap can be substantially hexagonal in configuration.

Broadly stated, in some embodiments, each of the plurality of compressible protrusions disposed on the wrap can comprise one or more of a group comprising rubber, thermoplastic rubber, foam rubber, polyurethane rubber, silicone rubber, thermoplastic resin and thermoplastic elastomer.

Broadly stated, in some embodiments, an insert pad can be provided for attaching to a garment, the insert pad comprising: a substrate; one or more protrusion pad disposed thereon, each comprising a plurality of compressible protrusions extending therefrom; and one or more attachment pad disposed thereon for attaching to the garment.

Broadly stated, in some embodiments, the substrate can comprise silicone rubber or neoprene.

Broadly stated, in some embodiments, wherein each of the plurality of compressible protrusions of the insert pad can comprise one or more of a group comprising rubber, thermoplastic rubber, foam rubber, polyurethane rubber, silicone rubber, thermoplastic resin and thermoplastic elastomer.

Broadly stated, in some embodiments, a dumbbell can be provided, comprising: a substantially straight handle segment disposed along a longitudinal axis between a pair of end faces; and a plurality of curved weight segments disposed along an arc path between the paid of end faces thereby enclosing the handle segment.

Broadly stated, in some embodiments, the handle segment can be rotatably attached to the dumbbell.

Broadly stated, in some embodiments, the handle segment can comprise an opening extending therethrough between the pair of end faces.

Broadly stated, in some embodiments, a strap can be disposed through the opening, the strap further comprising a ring for attachment to weight resistance exercise equipment.

Broadly stated, in some embodiments, the dumbbell can further comprise a barbell having first and second ends, the first end disposed in the opening of the dumbbell, the second end disposed in the opening of another of the dumbbell.

Broadly stated, in some embodiments, the dumbbell can further comprise an attachment mechanism disposed on one of both of the end faces, the attachment mechanism for attaching to weight resistance exercise equipment.

Broadly stated, in some embodiments, the dumbbell can further comprise an attachment mechanism disposed on one of more of the curved weight segments, the attachment mechanism for attaching to weight resistance exercise equipment.

Broadly stated, in some embodiments, the attachment mechanism can comprise a loop or a ring.

Broadly stated, in some embodiments, a galosh can be provided for footwear worn by a person during physical exercise, the galosh comprising: a sole comprising a plurality of compressible protrusions extending downwardly therefrom; a fastening mechanism for securing the galosh to the footwear; and a strap operatively coupled to the galosh, the strap further comprising a ring for attachment to weight resistance exercise equipment.

Broadly stated, in some embodiments, the fastening mechanism of the galosh can comprise a zipper.

Broadly stated, in some embodiments, each of the plurality of compressible protrusions of the galosh can comprise one or more of a group comprising rubber, thermoplastic rubber, foam rubber, polyurethane rubber, silicone rubber, thermoplastic resin and thermoplastic elastomer.

Broadly stated, in some embodiments, one or more of the plurality of compressible protrusions of the galosh can comprise an opening or void disposed therein.

Broadly stated, in some embodiments, the galosh can further comprise one or more weights for removably placing in the opening or void.

Broadly stated, in some embodiments, a foot attachment device can be provided for use with a weight resistance machine, the foot attachment device comprising: a footboard; a sole disposed on a bottom surface of the footboard, the sole comprising a plurality of compressible protrusions extending downwardly therefrom; an attachment mechanism for attaching the footboard to a foot of a person or to footwear worn by the person; and a strap operatively coupled to the footboard, the strap further comprising a ring for attachment to weight resistance exercise equipment.

Broadly stated, in some embodiments, wherein each of the plurality of compressible protrusions can comprise one or more of a group comprising rubber, thermoplastic rubber, foam rubber, polyurethane rubber, silicone rubber, thermoplastic resin and thermoplastic elastomer.

Broadly stated, in some embodiments, wherein one or more of the plurality of compressible protrusions can comprise an opening or void disposed therein.

Broadly stated, in some embodiments, wherein the foot attachment device can further comprise one or more weights for removably placing in the opening or void.

Broadly stated, in some embodiments, an enclosure for exercise can be provided, comprising: a base, comprising one or more first attachment member for attaching a first resistance or elastic band thereto; two sidewalls extending upwards from the base, one or both of the two sidewalls comprising one or more second attachment member for attaching a second resistance or elastic band thereto; a rear wall extending upwards from the base disposed between the two sidewalls, the rear wall comprising one or more third attachment member for attaching a third resistance or elastic band thereto; and a roof member disposed between the two sidewalls, the roof member extending from upper edges of the two sidewalls, the roof member comprising one or more fourth attachment member for attaching a fourth resistance or elastic band thereto.

Broadly stated, in some embodiments, a hand weight can be provided, comprising a compressible handgrip, further comprising a weight disposed therein.

Broadly stated, in some embodiments, the hand weight can further comprise a plurality of compressible protrusions extending outwardly therefrom wherein each of the plurality of compressible protrusions comprises one or more of a group comprising rubber, thermoplastic rubber, foam rubber, polyurethane rubber, silicone rubber, thermoplastic resin and thermoplastic elastomer.

Broadly stated, in some embodiments, wherein the plurality of compressible protrusions can comprise hemispherical-shaped protrusions.

Broadly stated, in some embodiments wherein the plurality of compressible protrusions can comprise hexagonal-shaped protrusions.

Broadly stated, in some embodiments, an exercise weight can be provided, comprising: a weight member; and a hook and loop fastener disposed on one surface of the weight member, the hook and loop fastener for attaching to a surface.

Broadly stated, in some embodiments, the surface can comprise clothing worn by a person exercising.

Broadly stated, in some embodiments, the surface can comprise an insole for a shoe.

### BRIEF DESCRIPTION OF THE DRAWINGS:

FIG. 1 is a top plan view depicting one embodiment of an exercise mat comprising compressible hexagonal protrusions.

FIG. 2A is a perspective view depicting a person exercising on the exercise mat of FIG. 1.

FIG. 2B is a perspective view depicting a person placing their hand on the exercise mat of FIG. 1.

FIG. 2C is a perspective view depicting a person placing their hand and knee on the exercise mat of FIG. 1.

FIG. 2D is a perspective view depicting the exercise mat of FIG. 1.

FIG. 3 is a side, close-up perspective view depicting one embodiment of a yoga mat comprising compressible hexagonal protrusions in a first pattern.

FIG. 4 is a front, close-up perspective plan view depicting the yoga mat of FIG. 3.

FIG. 5 is a perspective view depicting the yoga mat of FIG. 3.

FIG. 6 is a perspective view depicting a second embodiment of a yoga mat comprising compressible protrusions in a second pattern.

FIG. 7 is a side elevation view depicting Section Lines A-A of FIG. 6.

FIG. 8 is a top plan view depicting a third embodiment of a yoga mat comprising interlocking edge profiles.

FIG. 9 is a top perspective view depicting one embodiment of a spinal rocker comprising compressible hexagonal protrusions disposed thereon.

FIG. 10 is a bottom perspective view depicting the spinal rocker of FIG. 9.

FIG. 11 is a perspective plan view depicting one embodiment of a spinal mat system comprising compressible hexagonal protrusions disposed thereon.

FIG. 12 is a top plan view depicting one embodiment of a mat with compressible hexagonal protrusions thereon for use with the spinal mat system of FIG. 11.

FIG. 13 is a side elevation view depicting one embodiment of a wrap for footwear comprising compressible hexagonal protrusions disposed thereon to contact the sole of the footwear.

FIG. 14 is a perspective view depicting a hand grip being used by a person.

FIG. 15 is a top plan view depicting the hand grip of FIG. 14.

FIG. 16 is an elevation view depicting the inside of one embodiment of an abdominal exercise belt comprising compressible hexagonal protrusions disposed thereon.

FIG. 17 is an elevation view depicting the outside of the abdominal belt of FIG. 16.

FIG. 18 is a front elevation view depicting the abdominal belt of FIG. 16 wrapped around the abdomen of a person.

FIG. 19 is a front elevation view depicting the abdominal belt of FIG. 18 being opened up.

FIG. 20 is an elevation view depicting the inside of one embodiment of an arm wrap band comprising compressible hexagonal protrusions disposed thereon.

FIG. 21 is an elevation view depicting the outside of the arm wrap band of FIG. 21.

FIG. 22 is a front elevation view depicting the arm wrap band of FIG. 22 wrapped around the arm of a person.

FIG. 23 is a perspective view depicting the arm wrap band of FIG. 22.

FIG. 24 is an elevation view depicting the inside of one embodiment of a leg wrap band comprising compressible hexagonal protrusions disposed thereon.

FIG. 25 is an elevation view depicting the outside of the leg wrap band of FIG. 24.

FIG. 26 is a front elevation view depicting the leg wrap band of FIG. 24 wrapped around the quadricep and calf of the leg of a person.

FIG. 27 is a perspective view depicting one embodiment of exercise shorts comprising an inner liner of compressible hexagonal protrusions disposed thereon.

FIG. 28 is a front elevation view depicting one embodiment of an exercise shirt comprising an inner liner of compressible hexagonal protrusions disposed thereon.

FIG. 29 is an elevation view depicting the outside of a second embodiment of an abdominal exercise belt comprising compressible hexagonal protrusions disposed thereon, the belt comprising an inflatable bladder disposed therein.

FIG. 30 is an elevation view depicting the inside of the abdominal belt of FIG. 29.

FIG. 31 is an elevation view depicting the outside of a second embodiment of an arm wrap band comprising compressible hexagonal protrusions disposed thereon, the arm wrap band comprising an inflatable bladder disposed therein.

FIG. 32 is an elevation view depicting the inside of the arm wrap band of FIG. 31.

FIG. 33 is an elevation view depicting the outside of a second embodiment of a leg wrap band comprising compressible hexagonal protrusions disposed thereon, the leg wrap band comprising an inflatable bladder disposed therein.

FIG. 34 is an elevation view depicting the inside of the leg wrap band of FIG. 33.

FIG. 35 is a perspective view depicting a second embodiment of exercise shorts comprising an inner liner of compressible hexagonal protrusions disposed thereon, the liner comprising an inflatable bladder disposed therein.

FIG. 36 is a front elevation view depicting a second embodiment of an exercise shirt comprising an inner liner of compressible hexagonal protrusions disposed thereon, the liner comprising an inflatable bladder disposed therein.

FIG. 37 is a rear elevation view depicting the exercise shirt of FIG. 36.

FIG. 38 is a side elevation cross-section view depicting the compressible protrusions for used with the embodiments disclosed herein.

FIG. 39A is a side elevation cross-section view depicting a first embodiment of the compressible protrusions of FIG. 38.

FIG. 39B is a perspective view depicting the first embodiment of the compressible protrusions of FIG. 39A.

FIG. 39C is a top plan view depicting the first embodiment of the compressible protrusions of FIG. 39A.

FIG. 40A is a side elevation cross-section view depicting a second embodiment of the compressible protrusions of FIG. 38.

FIG. 40B is a perspective view depicting the second embodiment of the compressible protrusions of FIG. 40A.

FIG. 40C is a top plan view depicting the second embodiment of the compressible protrusions of FIG. 40A.

FIG. 41A is a side elevation cross-section view depicting a third embodiment of the compressible protrusions of FIG. 38.

FIG. 41B is a perspective view depicting the third embodiment of the compressible protrusions of FIG. 41A.

FIG. 41C is a top plan view depicting the third embodiment of the compressible protrusions of FIG. 41A.

FIG. 42A is a side elevation cross-section view depicting a fourth embodiment of the compressible protrusions of FIG. 38.

FIG. 42B is a perspective view depicting the fourth embodiment of the compressible protrusions of FIG. 42A.

FIG. 42C is a top plan view depicting the fourth third embodiment of the compressible protrusions of FIG. 42A.

FIG. 43A is a side elevation cross-section view depicting a fifth embodiment of the compressible protrusions of FIG. 38.

FIG. 43B is a perspective view depicting the fifth embodiment of the compressible protrusions of FIG. 43A.

FIG. 43C is a top plan view depicting the fifth third embodiment of the compressible protrusions of FIG. 43A.

FIG. 44 is a side elevation view depicting a step up bench with its legs retracted.

FIG. 45 is a perspective view depicting the step up bench of FIG. 44 with its legs folded out.

FIG. 46 is a side view depicting the step up bench of FIG. 45 with its back rest in a raised position.

FIG. 47 is a perspective view depicting a fat hand grip having a plurality of compressible protrusions disposed thereon.

FIG. 48 is a perspective view depicting the fat hand grip of FIG. 47 disposed on a bar used with an exercise machine.

FIG. 49 is a perspective view depicting the fat hand grip of FIG. 47 disposed on a pull-up bar.

FIG. 50 is a top plan view depicting one embodiment of an attachable pad having a plurality of compressible protrusions disposed thereon.

FIG. 51 is a top plan view depicting an alternate embodiment of the attachable pad of FIG. 50.

FIG. 52 is an elevation view depicting one embodiment of a pant garment having the attachable pads of FIG. 50 or 51 disposed thereon.

FIG. 53a is an end elevation view depicting one embodiment of a dumbbell weight.

FIG. 53b is a side elevation view depicting the dumbbell weight of FIG. 53a.

FIG. 53c is a perspective view depicting the dumbbell weight of FIG. 53a.

FIG. 54 is a perspective view depicting the dumbbell weight of FIG. 53a having a strap disposed through a handle opening thereof.

FIG. 55 is a side elevation view depicting the dumbbell weight of FIG. 54.

FIG. 56 is a perspective view depicting one embodiment of a barbell having the dumbbell weight of FIG. 54 without the strap as weights to the barbell.

FIG. 57 is a left perspective view depicting the dumbbell weight of FIG. 54 without the strap.

FIG. 58 is an end elevation view depicting the dumbbell weight of FIG. 57.

FIG. 59 is a right perspective view depicting the dumbbell weight of FIG. 57.

FIG. 60 is a front perspective view depicting a second embodiment of the dumbbell weight of FIG. 54 with attachment rings on end surfaces thereof.

FIG. 61 is a perspective view depicting a person exercising with the dumbbell of FIG. 60.

FIG. 62 is a top plan view depicting one embodiment of straps for use with the dumbbell of FIG. 60.

FIG. 63 is a perspective view depicting a person using the dumbbell of FIG. 60 and the straps of FIG. 62 as a handle on an exercise machine.

FIG. 64 is a front perspective view depicting another embodiment of the dumbbell of FIG. 60 with attachment rings on the weight segments thereof.

FIG. 65a is a perspective view depicting a footwear galosh having compressible protrusions on a sole thereof.

FIG. 65b is a rear elevation view depicting the footwear galosh of FIG. 56a.

FIG. 66 is a perspective view depicting the footwear galosh of FIG. 56a having a weight for placement in openings disposed in a sole thereof.

FIG. 67 is a perspective view depicting another embodiment of the footwear galosh of FIG. 66 having multiple straps attached thereto.

FIG. 68a is a side elevation view depicting one embodiment of a device for attaching to footwear, wherein the device has straps for attachment to an exercise machine.

FIG. 68b is a top plan view depicting the device of FIG. 68a.

FIG. 68c is front elevation view depicting the strap attachment of the device of FIG. 68a.

FIG. 69a is a side elevation view depicting a second embodiment of the device of FIG. 68a.

FIG. 69b is a top plan view depicting the device of FIG. 69a.

FIG. 69c is a perspective view depicting the strap attachment of the device of FIG. 69a.

FIG. 70 is a perspective view depicting one embodiment of a cage for use with the dumbbell of FIGS. 60 and 64 in addition to the device of FIG. 68a)-c) and 69a)-c).

FIG. 71 perspective view depicting one embodiment of a cable exercise machine for use with the dumbbell of FIGS. 60 and 64 in addition to the device of FIG. 68a)-c) and 69a)-c).

FIG. 72 is a front perspective view depicting one embodiment of a hand weight having hemispherical compressible protrusions on an outer surface thereof.

FIG. 73 is front perspective view depicting a second embodiment of the hand weight of FIG. 72 having hexagonal compressible protrusions on an outer surface thereof.

FIG. 74 is a front perspective view depicting one embodiment of a hand weight having a handle with compressible protrusions on an outer surface thereof.

FIG. 75 is a front perspective view depicting a person exercising with the hand weights of FIG. 74.

FIG. 76 is a front perspective view depicting a weight for attachment to clothing using a hook and loop attachment mechanism.

FIG. 77 is a front perspective view depicting the weight of FIG. 76 for attachment to a glove garment.

FIG. 78 is a top plan view depicting a fourth embodiment of a yoga mat.

FIG. 79 is a front perspective view depicting a hex pad for use with the yoga mat of FIG. 78.

FIG. 80 is a front perspective view depicting an embodiment of a two-layer mat.

FIG. 81 is a front exploded view depicting the two-layer mat of FIG. 80.

FIG. 82 is a top perspective view depicting one embodiment of a receiver mat for the two-layer mat of FIG. 80.

FIG. 83 is a front perspective view depicting the receiver mat of FIG. 82.

FIG. 84 is a top plan view depicting one embodiment of compressible protrusions for attaching to the sole of a shoe.

FIG. 85 is a side elevation view depicting the compressible protrusions of FIG. 84.

### DETAILED DESCRIPTION OF EMBODIMENTS:

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology can include a variety of combinations and/or integrations of the embodiments described herein.

The presently disclosed subject matter is illustrated by specific but non-limiting examples throughout this description. The examples may include compilations of data that are representative of data gathered at various times during the course of development and experimentation related to the present invention(s). Each example is provided by way of explanation of the present disclosure and is not a limitation thereon. In fact, it will be apparent to those skilled in the art that various modifications and variations can be made to the teachings of the present disclosure without departing from the scope of the disclosure. For instance, features illustrated or described as part of one embodiment can be used with another embodiment to yield a still further embodiment.

All references to singular characteristics or limitations of the present disclosure shall include the corresponding plural characteristic(s) or limitation(s) and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

While the following terms used herein are believed to be well understood by one of ordinary skill in the art, definitions are set forth to facilitate explanation of the presently disclosed subject matter.

Unless defined otherwise, all technical and scientific terms used herein have the same meaning as commonly understood by one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims.

Unless otherwise indicated, all numbers expressing quantities, properties, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about”, when referring to a value or to an amount of mass, weight, time, volume, concentration or percentage is meant to encompass variations of in some embodiments +/−50%, in some embodiments +/−40%, in some embodiments +/−30%, in some embodiments +/−20%, in some embodiments +/−10%, in some embodiments +/−5%, in some embodiments +/−1%, in some embodiments +/−0.5%, and in some embodiments +/−0.1% from the specified amount, as such variations are appropriate to perform the disclosed method.

Alternatively, the terms “about” or “approximately” can mean within an acceptable error range for the particular value as determined by one of ordinary skill in the art, which will depend in part on how the value is measured or determined, i.e., the limitations of the measurement system. For example, “about” can mean within 3, or more than 3, standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Unless otherwise indicated, all numbers expressing quantities, properties, and so forth used in the specification and claims are to be understood as being modified in all instances by the term “about”. And so, the numerical parameters set forth in this specification and claims are approximations that can vary depending upon the desired properties sought to be obtained by the presently disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

In some embodiments, a compressible protrusion can be provided. The compressible protrusion can comprise a planar surface and a protrusion from said planar surface. The compressible protrusion can be configured to be disposed between a person and a piece of exercise equipment or any hard surface. The compressible protrusion can be configured such that it can be attached to a shoe, glove, garment, sock, shoe cover or other exercise apparatus.

In some embodiments, the compressible protrusion can comprise a hexagonal configuration whereby the spacing between the hexagonal compressible protrusions can comprise a substantially uniform width channel therebetween.

In some embodiments including those described herein, the compressible protrusions can improve the user's body positioning when performing exercises or other activities. The compressible protrusions can cause the user to actively hold the muscles, joints and body in a proper alignment providing proprioceptive feedback to the CNS (the “proprioceptive effect”). These voluntary movements can be repeated and stored as central commands. This patterning of the brain allows various skills to be performed without continuous without continuous reference to consciousness and can increase the conscious and unconscious awareness of the joint or limb in motion.

In some embodiments, including those described herein, the compressible protrusions can be comprised of an elastomeric material that has physical properties that enable it to be compressed under force and to substantially return to its original shape when the force is released. Such materials can include rubber, thermoplastic rubber, foam rubber, polyurethane rubber, silicone rubber, thermoplastic resin, thermoplastic elastomers and any other material having similar physical properties as well known to those skilled in the art. In further embodiments, the exterior of the compressible hemisphere can be composed of a durable material, such as plastic or synthetic fiber and the interior of the compressible protrusion can comprise one or more of a liquid, a gel and a gas, such as air.

In some embodiments, the compressible protrusions can comprise a low density that allows them to be compressed or be deformed so as to provide the proprioceptive effect to a person's body when exercising. In some embodiments, the compressible protrusions can comprise a high density that prevents or resists compression or deformation so as to provide an acupressure effect to a person's body when exercising.

Though the compressible protrusions in this application are at times described as hexagons, for the purposes of this specification and the claims herein, it shall be understood that any compressible protrusion described as a hemisphere or as being hemispherical can be substituted with other shapes such as a hemi-ovoid, hemi-ellipsoid, tetrahedron, trapezoidal prism or other similarly curved or polyhedral surface, or other suitable shape as well known to those skilled in the art.

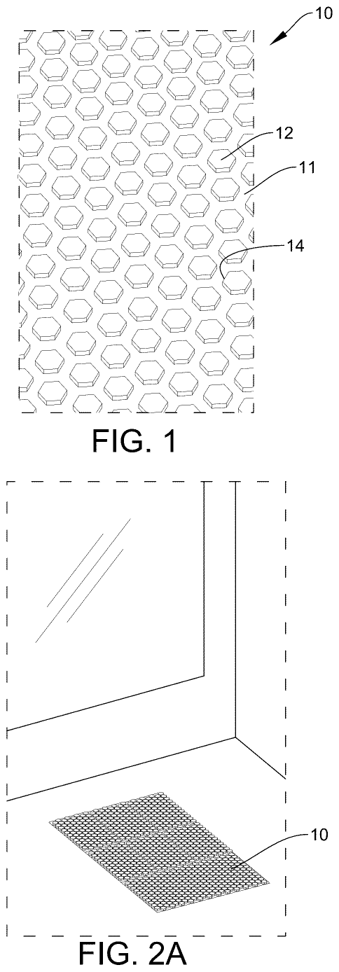

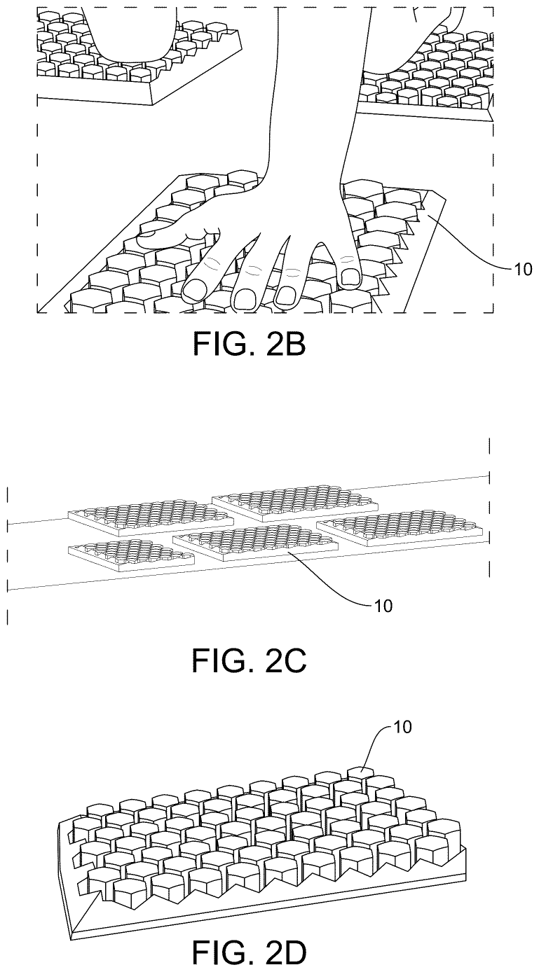

In some embodiments, an exercise mat can be provided. Referring to FIGS. 1 and 2, one embodiment of mat 10 is shown. In some embodiments, mat 10 can comprise a plurality of spaced-apart hexagonal compressible protrusions 12 disposed thereon. In some embodiments, protrusions 12 can be spaced apart to form channel 14 therebetween wherein channel 14 can be substantially uniform in width throughout, which can provide sufficient space for protrusions 12 to expand into as it is deformed when weight is applied thereon to compress protrusions 12 causing them to decrease in height but increase in width. In some embodiments, mat 10 can be approximately 8 mm thick where base layer 11 can be approximately 4 mm thick and the height of protrusions 12 being approximately 4 mm.

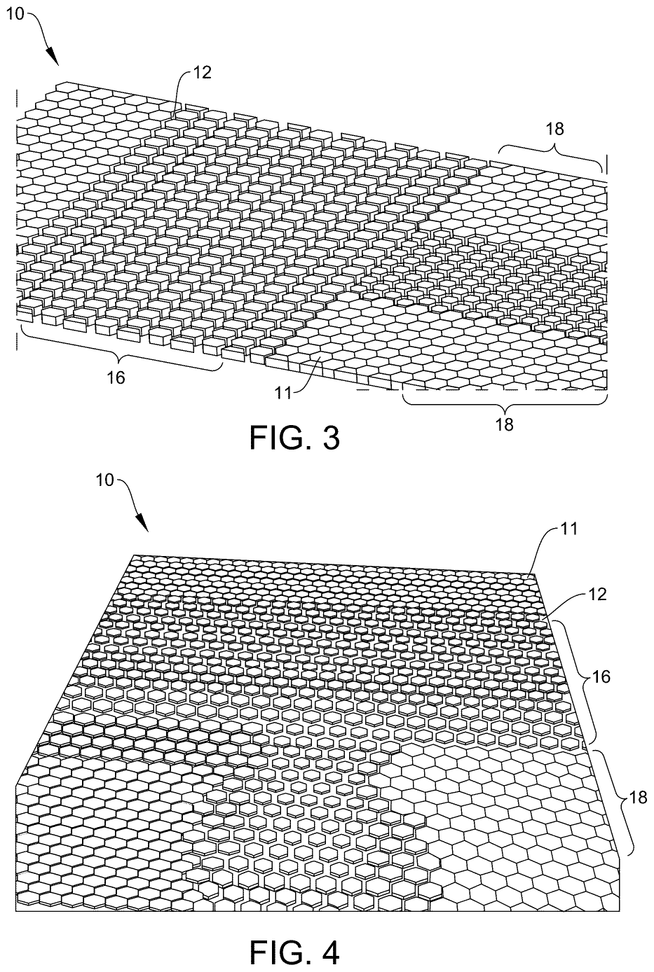

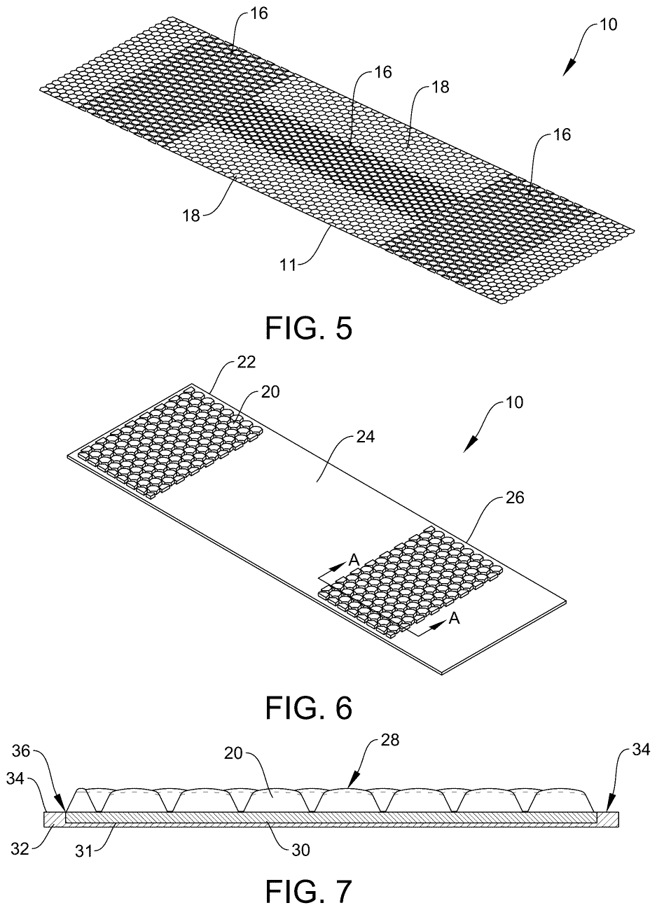

In some embodiments, exercise mat 10 can be configured as a yoga mat, as shown in FIGS. 3 to 5. In some embodiments, mat 10 can comprise protrusions 12 that have differing heights as well as differing inclinations in different regions of mat 10. This can help create unevenness and a small “uncertainty” factor due to reduced surface area on the top surface of mat 10. In some embodiments, protrusions 12 can comprise varying heights in various regions across mat 10, on either side of mat 10, that can increase instability and, thus, improve workouts carried out on mat 10. In some embodiments, mat 10 can comprise protrusions 12 disposed in locations 16 whereas regions 18 can be devoid of protrusions 12. In some embodiments, base layer 11 can be approximately 4 mm in thickness whereas the height of protrusions 12 can range from approximately 1 mm to 4 mm.

Referring to FIGS. 6 and 7, an alternate embodiment of mat 10 configured as a yoga mat is shown. In some embodiments, mat 10 can comprise of head region 22, waist region 24 and foot region 26 with compressible protrusions 20 disposed in head and foot regions 22 and 26. In some embodiments, mat 10 can comprise of a multi-layer construction. In some embodiments, protrusions 20 can be disposed on protrusion layer 30, which can be comprised of nitrile butadiene rubber (“NBR”) foam. In some embodiments, mat 10 can comprise base layer 32 with top layer 34 disposed thereon. One or both of base layer 32 and top layer 34 can be comprised of NBR foam. In some embodiments, mat 10 can comprise of depression or relief area 31 wherein protrusion layer 30 can be disposed therein and then can be further secured with stitching 36. In some embodiments, protrusions 20 can be covered by layer 28 that can comprise lycra or other similar material to provide additional comfort for the user.

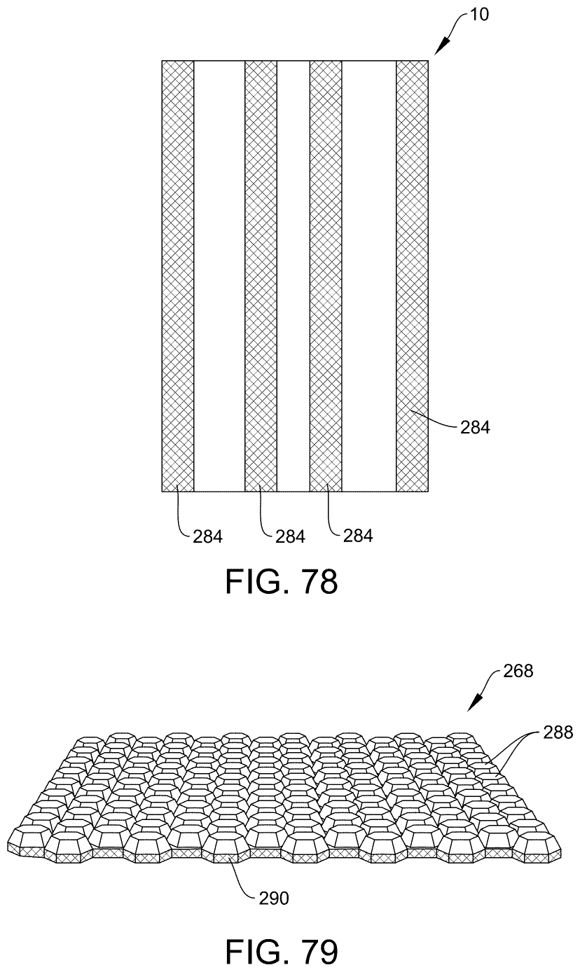

Referring to FIG. 78, a further embodiment of yoga mat 10 is shown. In this illustrated embodiment, yoga mat 10 can be comprised of rubber, foam rubber, neoprene, polyvinylchloride or other material as well known by those skilled in the art wherein yoga mat 10 can further comprise of one or more strips 284 for use with a loop and hook fastening mechanism. In some embodiments, strips 284 can be comprised of hooks for a loop and hook fastening mechanism whereas in other embodiments, strips can be comprised of loops for a loop and hook fastening mechanism. Referring to FIG. 79, one embodiment of hex pad 286 for use with yoga mat 10 of FIG. 78 is shown. In some embodiments, hex pad 286 can comprise a plurality of compressible protrusions 288 disposed on a top surface thereof, which can be similar to form and function of the compressible protrusions disclosed within this application. In some embodiments, hex pad 286 can further comprise of fastener layer 290 disposed on a bottom surface thereof. In some embodiments, layer 290 can be for use with a loop and hook fastening mechanism. In some embodiments, layer 290 can be comprised of hooks for a loop and hook fastening mechanism whereas in other embodiments, layer 290 can be comprise of loops for a loop and hook fastening mechanism. For example, if strips 284 comprise loops for a loop and hook fastening mechanism, layer 290 can comprise hooks for a loop and hook fastening mechanism thereby allowing hex pad 286 to be placed in any position on top of strips 284 of yoga mat 10 to allow a person exercising on yoga mat 10 to have hex pads 286 placed beneath body where the proprioceptive effect caused by compressible protrusions 288 is desired.

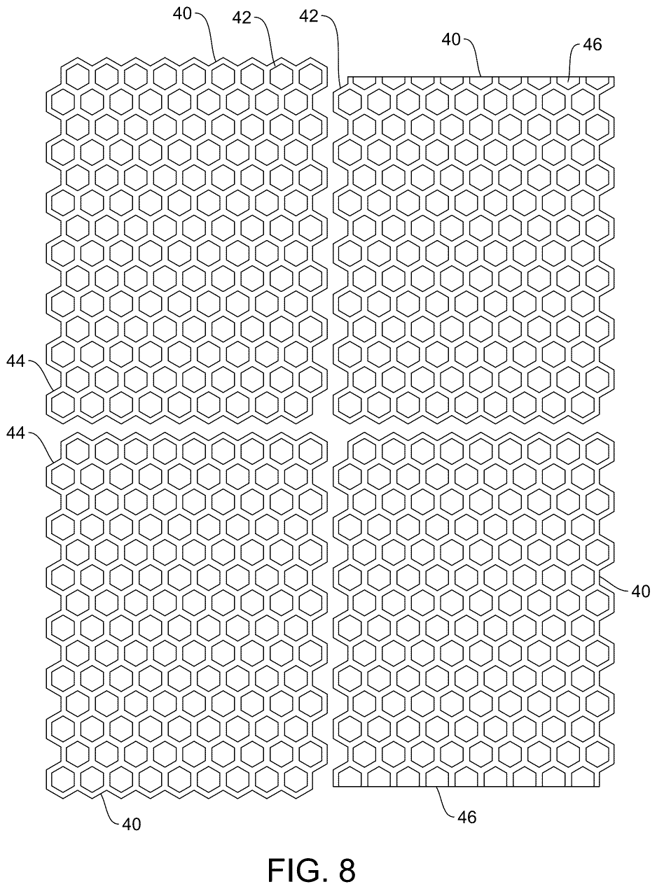

In some embodiments, mat 10 can be comprised of multiple pieces thereof that can be assembled into a larger mat, as shown in FIG. 8. In some embodiments, mat 10 can comprise of a first interlocking edge 42 disposed therealong one side thereof so that adjacent pieces of mat 10 can be closely fitted together. In the illustrated embodiment, edges 42 can comprise a zig-zag pattern following along 3 sides of the hexagonal pattern of the compressible protrusions. In some embodiments, mat 10 can comprise of a second interlocking edge 44 disposed therealong sides thereof that are perpendicular to edges 42 so that adjacent pieces of mat 10 can be closely fitted together. In the illustrated embodiment, edges 44 can comprise a sawtooth pattern following along 2 sides of the hexagonal pattern of the compressible protrusions.

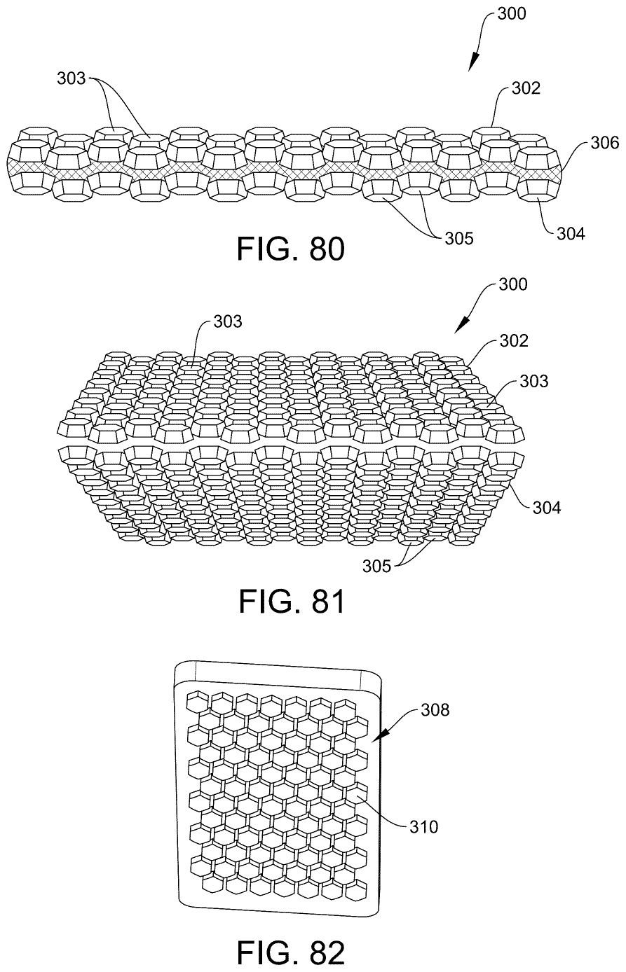

Referring to FIGS. 80 and 81, illustrated is one embodiment of two-layer mat 300 that can comprise of first layer 302 disposed on a top surface thereof, and of second layer 304 disposed on a bottom surface thereof. In some embodiments, first layer 302 can comprise a plurality of compressible protrusions 303, and second layer 304 can comprise a plurality of compressible protrusions 305. In some embodiments, compressible protrusions 303 can comprise of low density elastomeric material, as described above, whereas compressible protrusions 305 can comprise of high density elastomeric material, as described above. In some embodiments, first and second layers 302 and 304 can be adjoined along seam 306. In some embodiments, first and second layers 302 and 304 can be joined together with adhesive whereas in other embodiments, first and second layers 302 and 304 can be molded together. In some embodiments, first layer 302 can be used to provide a proprioceptive effect when stood on or laid upon by a person performing physical exercise. In some embodiments, second 304 can be used to provide an acupressure effect when stood on or laid upon by a person performing physical exercise.

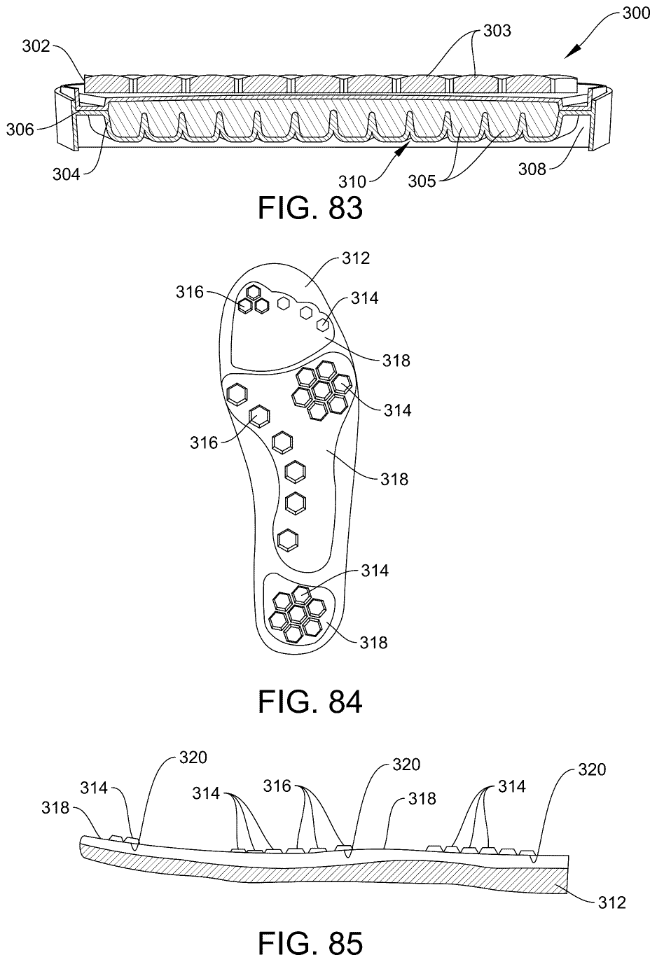

Referring to FIGS. 82 and 83, one embodiment of receiver mat 308 is shown. In some embodiments, receiver mat 308 can be comprised of high density elastomeric material, as described above, and can further comprise a plurality of depressions 310. In some embodiments, depressions 310 can be configured to receive compressible protrusions 305 of second layer 304 or to receive compressible protrusions 303 of first layer 302 wherein receiver mat 308 can be placed on a chair, exercise bench, or on a seat of an exercise machine with the compressible protrusions of two-layer mat 300 fitted into depressions 310 wherein a person can sit or stand therein while exercising.

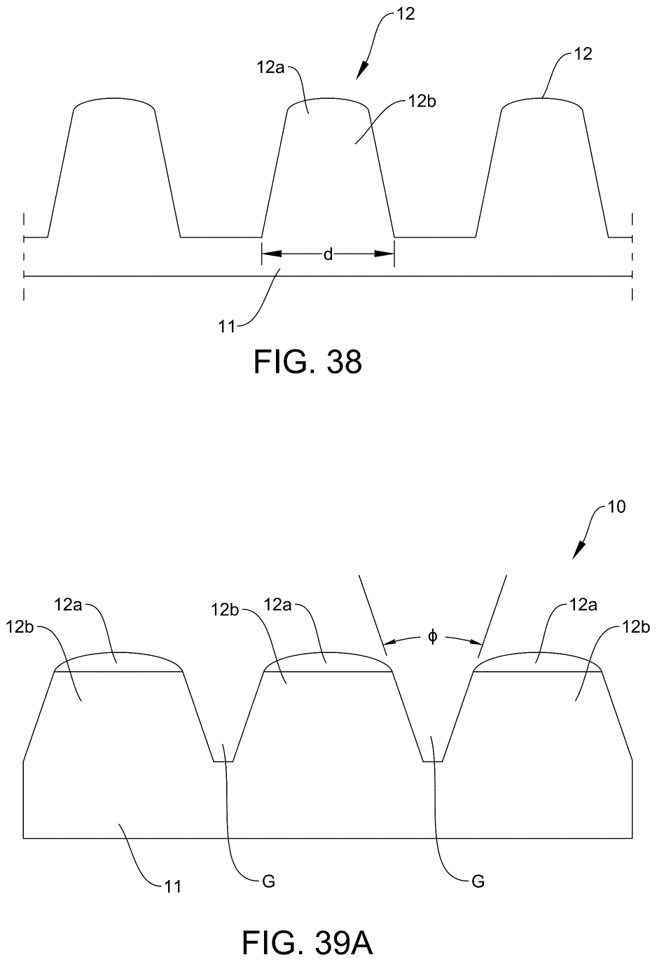

Referring to FIG. 38 to FIG. 43C, various embodiments of compressible protrusion 12 are shown, which show the physical configuration and characteristics of compressible protrusion 12 that can affect instability. FIG. 38 shows the subcomponents of compressible protrusion 12, which can be comprised of core portion 12b disposed on base layer 11. Core portion 12b, in some embodiments, can further comprise curved dome portion 12a disposed on top thereof. As shown in FIG. 38, core portion 12b can have a height defined as “b”, whereas dome portion 12a can have a height defined as “a”, thereby resulting in compressible protrusion 12 having a height defined as “H =a +b”. Protrusion 12 can further comprise a diameter “d” as defined by the width of the base of core portion 12b. The space or gap between adjacent protrusions 12 is labelled as “G” and can be defined as the distance between the bases of adjacent core portions 12b. In embodiments comprising dome portion 12a, the percentage of dome portion 12a making up height H is defined as “a/(a +b)” or “a/H”. In some embodiments, the sides of core portion 12b can be inclined such that an angle phi, “+”, is deposed between adjacent protrusions 12.

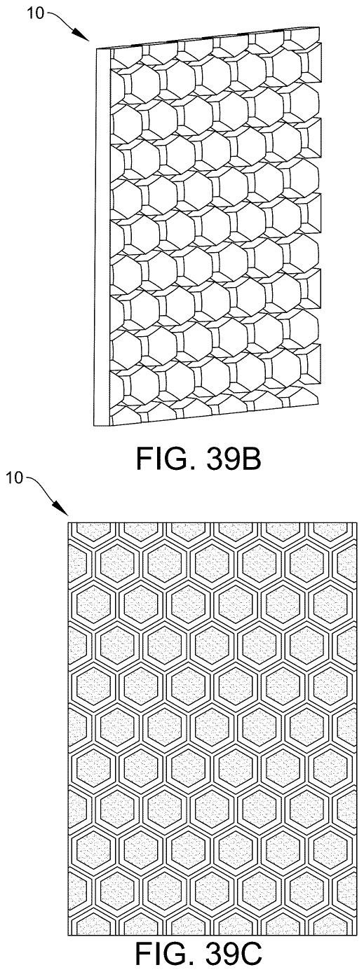

Referring to FIG. 39A, a first embodiment of compressible protrusion 12 disposed on mat 10 is shown on base layer 11 having a thickness of approximately 3.5 mm, where protrusion 12 can comprise a diameter of approximately 18 mm, and where core portion 12b can comprise a height of approximately 12 mm. Dome portion 12a can comprise a dome percentage of approximately 30%. The space, G, between adjacent protrusions can be approximately 1 mm. The inclination angle, phi, between adjacent protrusions can be approximately 20°. FIGS. 39B and 39C show perspective and top plan views, respectively, of this first embodiment.

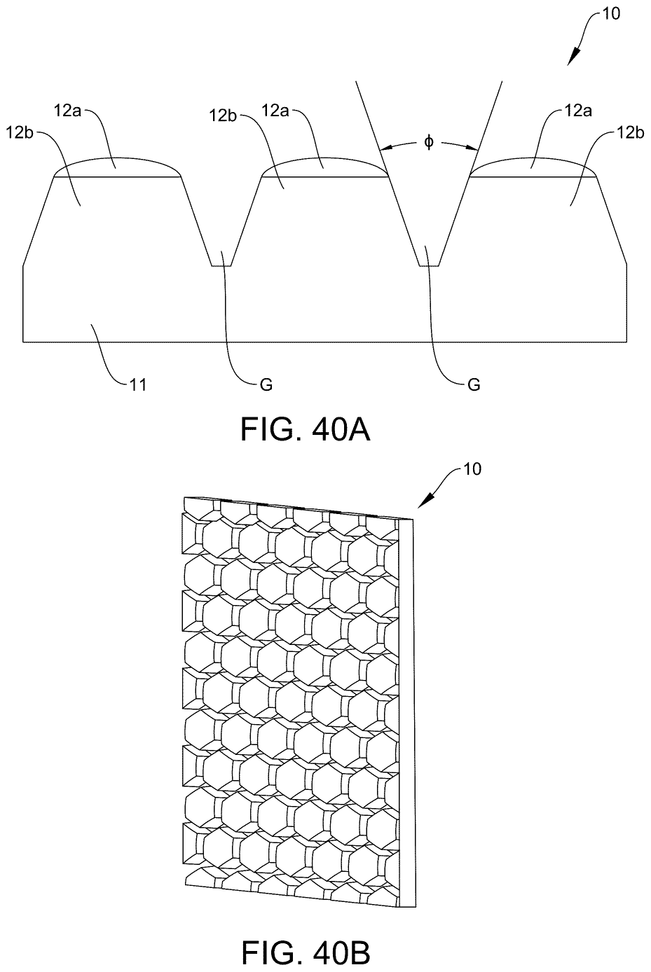

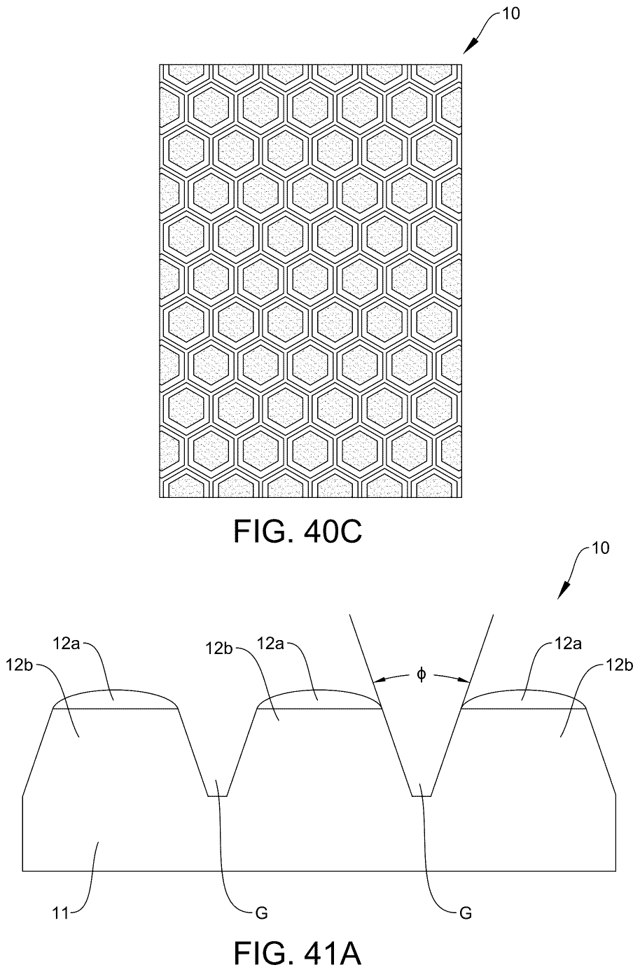

Referring to FIG. 40A, a second embodiment of compressible protrusion 12 disposed on mat 10 is shown on base layer 11 having a thickness of approximately 3.5 mm, where protrusion 12 can comprise a diameter of approximately 18 mm, and where core portion 12b can comprise a height of approximately 8 mm. Dome portion 12a can comprise a dome percentage of approximately 40%. The space, G, between adjacent protrusions can be approximately 1.5 mm. The inclination angle, phi, between adjacent protrusions can be approximately 20°. FIGS. 40B and 40C show perspective and top plan views, respectively, of this second embodiment.

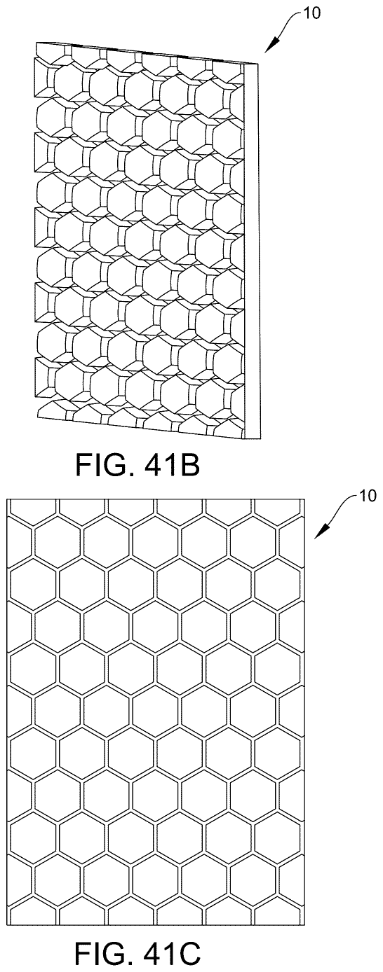

Referring to FIG. 41A, a third embodiment of compressible protrusion 12 disposed on mat 10 is shown on base layer 11 having a thickness of approximately 3.5 mm, where protrusion 12 can comprise a diameter of approximately 18 mm, and where core portion 12b can comprise a height of approximately 6 mm. Dome portion 12a can comprise a dome percentage of approximately 50%. The space, G, between adjacent protrusions can be approximately 2.5 mm. The inclination angle, phi, between adjacent protrusions can be approximately 10°. FIGS. 41B and 41C show perspective and top plan views, respectively, of this third embodiment.

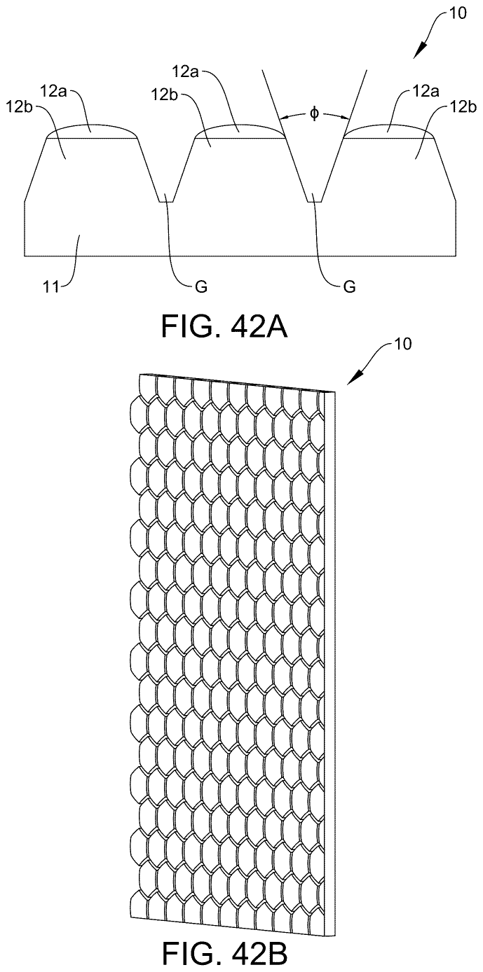

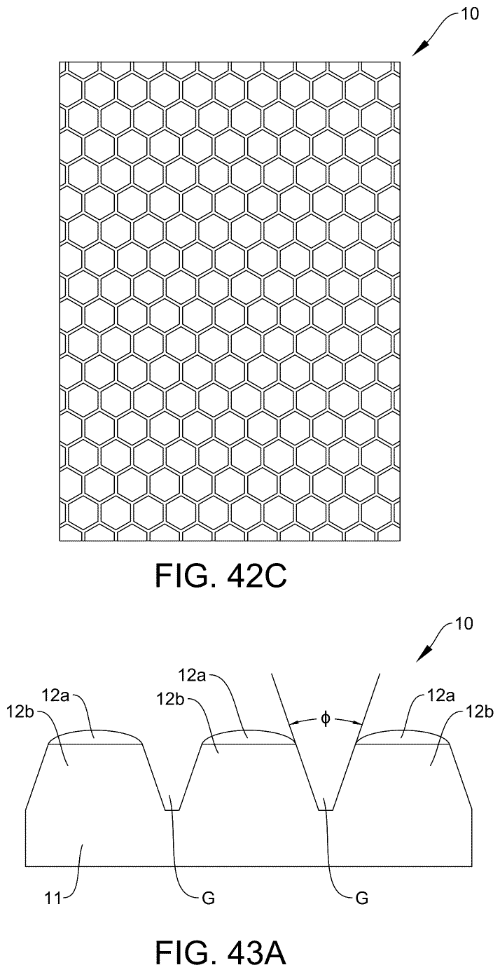

Referring to FIG. 42A, a fourth embodiment of compressible protrusion 12 disposed on mat 10 is shown on base layer 11 having a thickness in a range of approximately 2 to 3.5 mm, where protrusion 12 can comprise a diameter of approximately 10 mm, and where core portion 12b can comprise a height of approximately 4 mm. Dome portion 12a can comprise a dome percentage of approximately 70%. The space, G, between adjacent protrusions can be approximately 1.5 mm. The inclination angle, phi, between adjacent protrusions can be approximately 20°. FIGS. 42B and 42C show perspective and top plan views, respectively, of this fourth embodiment.

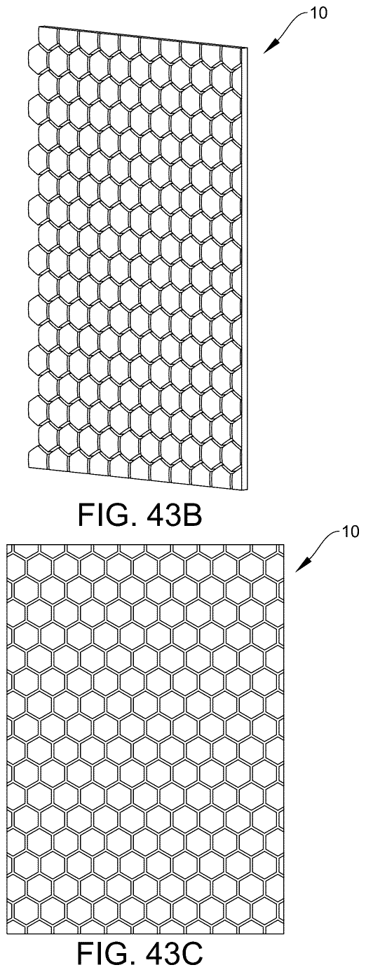

Referring to FIG. 43A, a fifth embodiment of compressible protrusion 12 disposed on mat 10 is shown on base layer 11 having a thickness in a range of approximately 2 to 3.5 mm, where protrusion 12 can comprise a diameter of approximately 10 mm, and where core portion 12b can comprise a height of approximately 2 mm. Dome portion 12a can comprise a dome percentage of approximately 70%. The space, G, between adjacent protrusions can be approximately 1.5 mm. The inclination angle, phi, between adjacent protrusions can be approximately 20°. FIGS. 43B and 43C show perspective and top plan views, respectively, of this fifth embodiment.

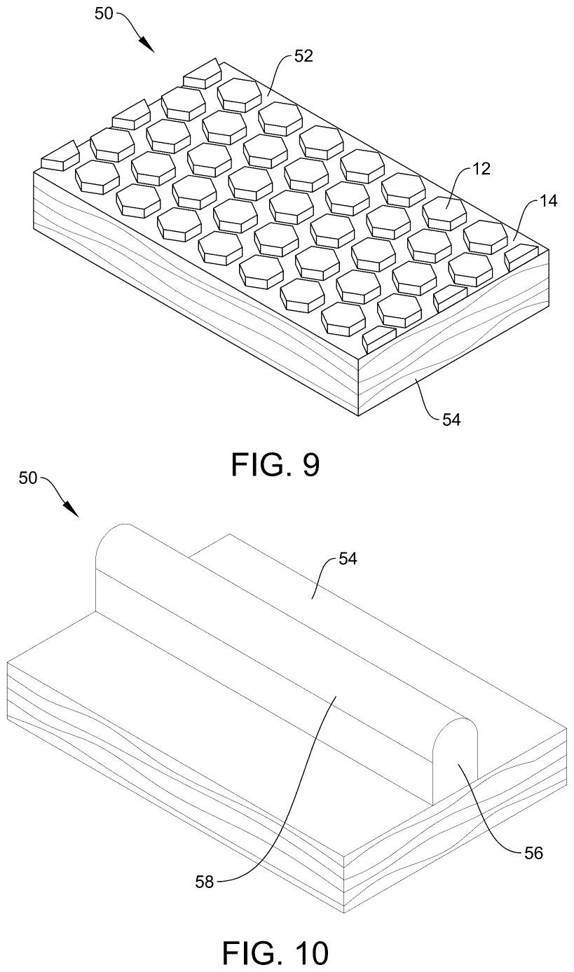

Referring to FIGS. 9 and 10, one embodiment of spinal rocker 50 is shown. In some embodiments, spinal rocker 50 can be used as a wellness tool to mobilize and to improve the range of motion of the lumbar, thoracic, and cervical spine as well as sacroiliac joints. Spinal rocker 50 can reproduce movements in the normal gait pattern in terms of flexion, extension, and rotation. In some embodiments, spinal rocker 50 can be moved up and down the spine in a horizontal or vertical position for the purpose of improving movement and blood flow. In some embodiments, spinal rocker can comprise of base plate 54, which can be made of wood or other solid material, with mat 52 disposed on a top surface thereof wherein mat 52 can comprise a plurality of compressible protrusions 12 thereon having channels 14 therebetween, as described above. In some embodiments, spinal rocker 50 can further comprise spine 56 disposed on a bottom surface of base plate 54, wherein spine 56 can comprise of semi-circular tread 58 disposed thereon to facilitate the rocking motion when placed on a floor surface.

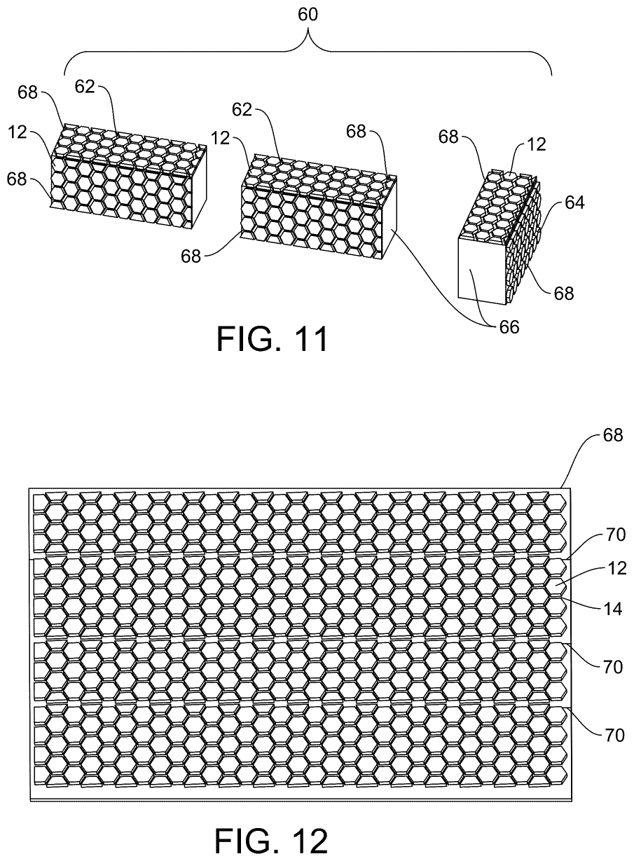

Referring to FIGS. 11 and 12, one embodiment of a spine mat system 60 is shown. In some embodiments, system 60 can comprise of one or more spine blocks 62 and a hip box 64 wherein spine blocks 62 can be configured to align with and support a person's spine and hip box 64 can be configured to align with and support the person's hips when the person lies down on blocks 62 and 64. In some embodiments, one or both of blocks 62 and 64 can comprise form core 66, which can be comprised of ethylene-vinyl acetate or some other functionally equivalent material as well known to those skilled in the art. In some embodiments, one of both of blocks 62 and 64 can be covered a plurality of hexagonal compressible protrusions 12. In some embodiments, one or both of blocks 62 and 64 can be covered with mat 68, as shown in FIG. 12, which can comprise a plurality of hexagonal compressible protrusions 12 disposed thereon separated by channels 14. In some embodiments, the width of one or both of blocks 62 and 64 can correlate approximately to the width of a person's spine so as to increase instability and, thus, improve workouts carried out on spine mat system 60. In some embodiments, the width of one or both of blocks 62 and 64 can be in the range of 50 mm to 200 mm whereas in representative embodiments, the width can be in the range of 75 mm to 100 mm. In some embodiments, mat 68 can further comprise crease or fold lines 70 that enable mat 68 to wrap around one or both of blocks 62 and 64 and be attached thereto with fasteners, adhesive or other functionally equivalent attachment means as well known to those skilled in the art.

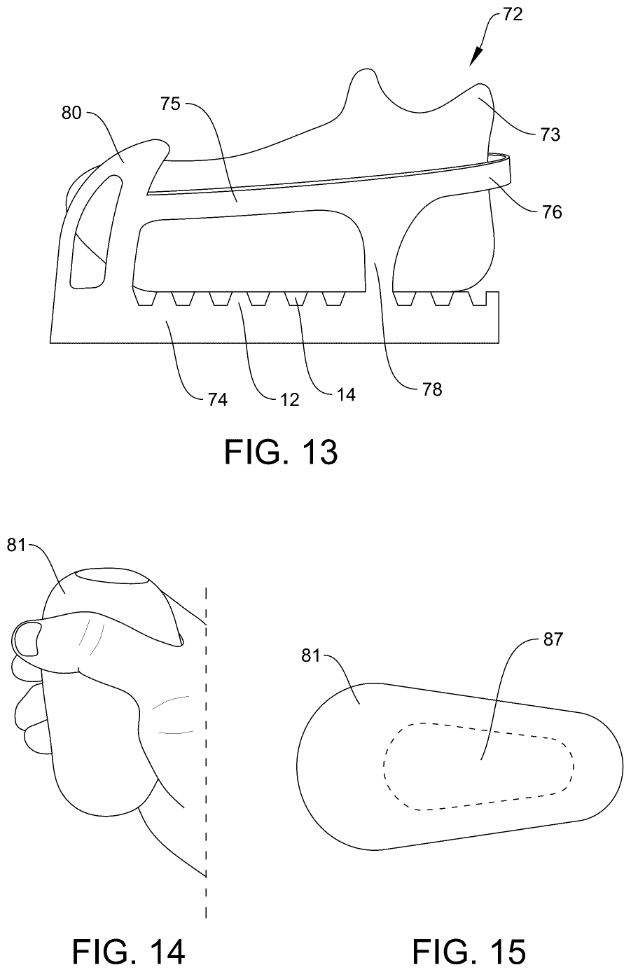

Referring to FIG. 13, one embodiments of hex shoe mat 72 is shown, that can be slipped onto a person's shoe to provide a proprioceptive effect as the person stands or moves during exercise movements. In some embodiments, shoe mat 72 can comprise of sole 74, toe piece 80 attached thereto, harness 75, heel loop 76 and sole connector 78. In some embodiments, sole 74 can comprise a plurality of hexagonal compressible protrusions 12, separated by channels 14, disposed on a top surface thereof and configured to contact the bottom of shoe 73. In some embodiments, hex shoe mat 72 can be comprised of one or more of silicone, natural rubber, synthetic rubber and any other functionally equivalent elastomeric material as well known to those skilled in the art so as to enable the person to easily slip shoe mat 72 on and off their shoe.

Referring to FIGS. 14 and 15, one embodiment of compressible hand weight 81 is shown, which can improve motion mechanics for a person as perform an exercise by squeezing hand weight 81. Doing so can enhance running and exercise forms to their most efficient state. Squeezing hand weight 81 during exercise can instill a connection between the arms, core and legs while increasing intensity and enhancing efficiency. Hand weight 81 can be comprised of silicone, natural rubber, synthetic rubber and any other functionally equivalent elastomeric material as well known to those skilled in the art so as to enable the person to compress hand weight 81 during exercise. In some embodiments, hand weight 81 can comprise of weight 87 disposed therein to add resistance in arm movements and to further enhance the effects of exercise. In some embodiments, weight 87 can be comprised of metal or other dense and material suitable for exercise weight equipment as well known to those skilled in the art. In some embodiments, hand weight 81 can comprising a plurality of compressible prot

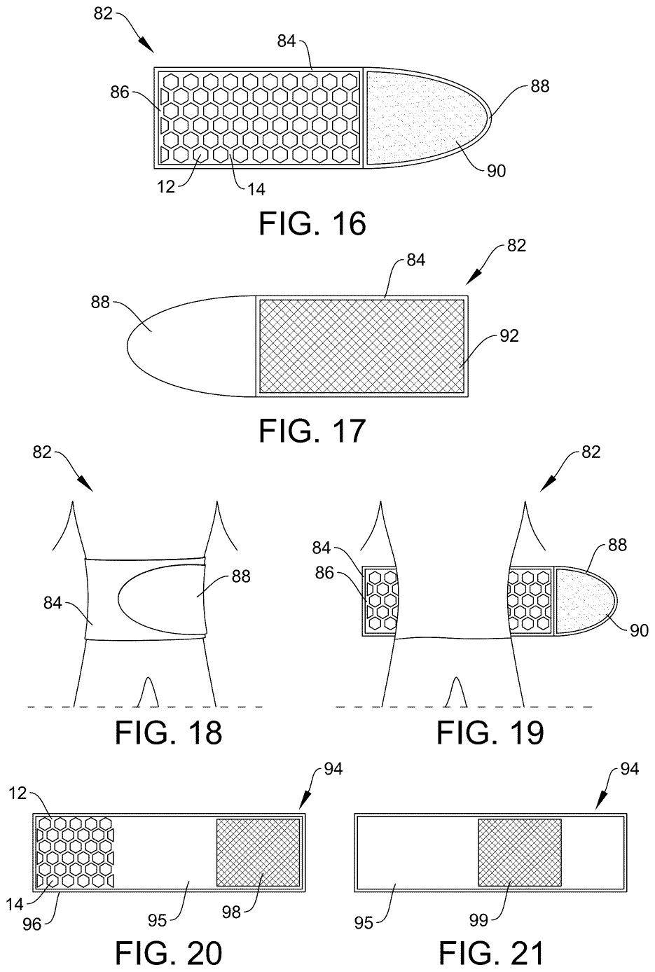

Referring to FIGS. 16 to 19, one embodiment of exercise belt 82 is shown. In some embodiments belt 82 can comprise of waist band 84 and attachment portion 88 attached to one thereof and configured to wrap around the abdomen of a person. In some embodiments, belt 82 can be comprised of one or more of spandex, silicone, latex rubber, synthetic rubber, neoprene and any other functionally equivalent material as well known to those skilled in the art. In some embodiments, belt 82 can comprise a hook and loop fastening mechanism, such as Velcro®, to hold belt 82 around the person. In some embodiments, attachment portion 88 can comprise of hook pad 90 disposed on an inward facing surface of and configured to attach to loop pad 92 disposed on an outwardly facing surface of band 84. In some embodiments, band 84 can comprise hex pad 86 disposed on an inwardly facing surface thereof wherein hex pad 86 can comprise a plurality of hexagonal compressible protrusions 12 disposed thereon, separated by channels 14. In some embodiments, hex pad 86 can be sewn to band 84 whereas in other embodiments, hex pad 86 can releasably attached to band 84 using a hook and loop mechanism to allow hex pad 86 to be positioned anywhere along band 84 as desired. When belt is wrapped around the abdomen of the person, the addition of hexagonal compressible protrusions 12 pressed against the person can induce a proprioceptive effect on exercise carried out by the person.

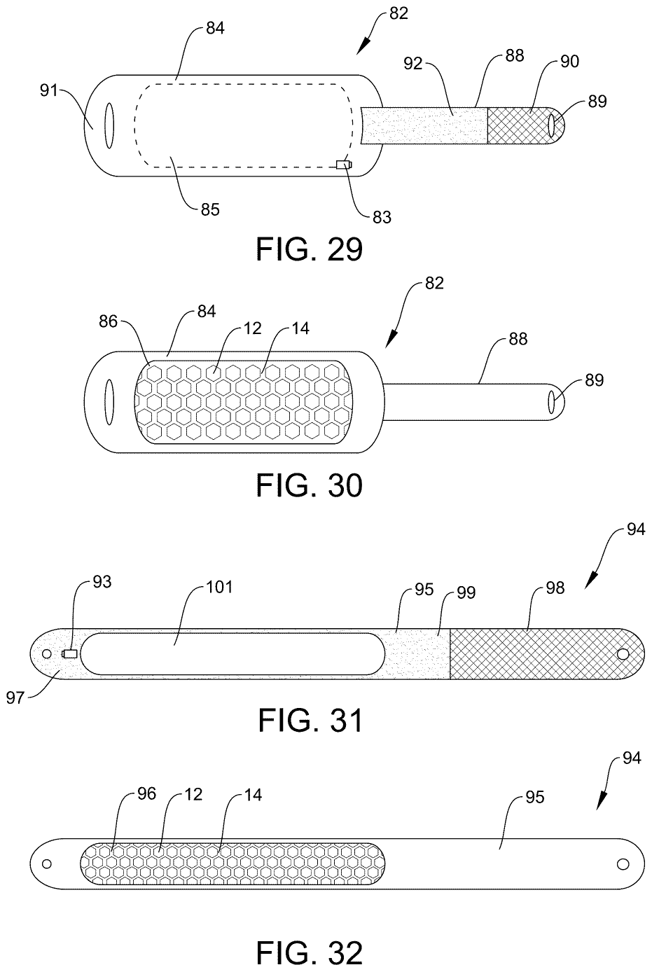

Referring to FIGS. 29 and 30, an alternate embodiment of belt 82 is shown. In this embodiment, band 84 can comprise inflatable air bladder 85 disposed therein. Air bladder 85 can be inflated by pumping air in through inflation valve 83, as well known to those skilled in the art. In this embodiment, attachment portion 88 can comprise of pull tab 89 that can be pulled through loop 91 to attach belt 84 to the person. Attachment portion 88 can further comprise hook pad 90 and loop pad 92 disposed thereon such that hook pad 90 can attach to loop pad 92 after passing through loop 91. When air bladder 85 is inflated, the proprioceptive effect of hex pad 86 can be accentuated as the person exercises.

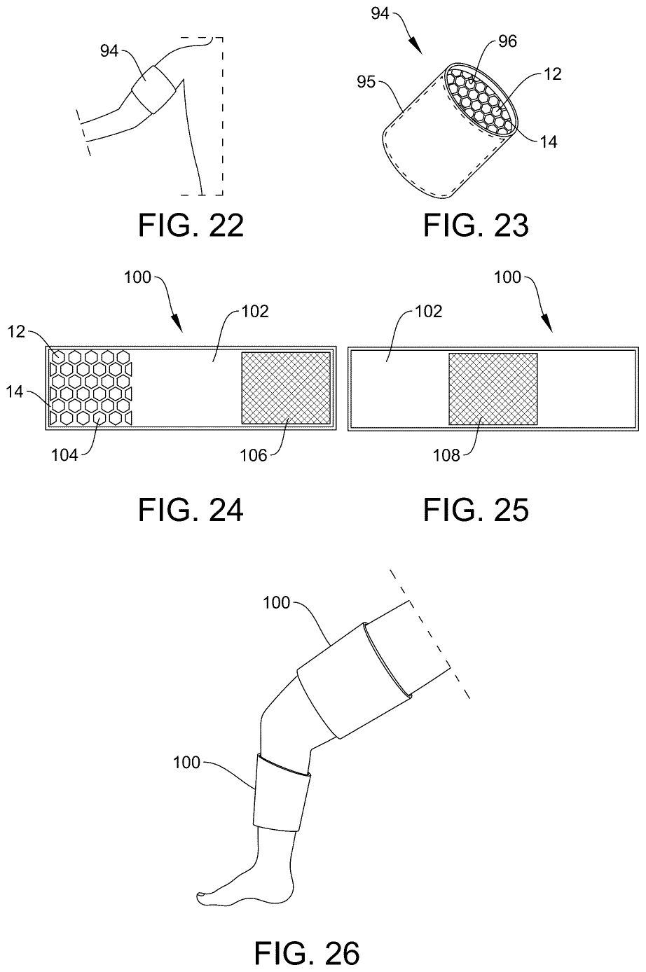

Referring to FIGS. 20 to 23, one embodiment of arm wrap band 94 is shown. In some embodiments, arm wrap band 94 can comprise of band 95 further comprising of hex pad 96 and hook pad 98 disposed on an inwardly facing surface thereof, and loop pad 99 disposed on an outwardly facing surface thereof. In some embodiments, arm wrap band 94 can be comprised of one or more of spandex, silicone, latex rubber, synthetic rubber, neoprene and any other functionally equivalent material as well known to those skilled in the art. In some embodiments, hex pad 96 can comprise a plurality of hexagonal compressible protrusions 12, separated by channels 14, disposed thereon. In some embodiments, band 95 can be comprised of neoprene or some other functionally equivalent material as well known to those skilled in the art. When arm wrap band 94 is wrapped around an arm of a person, hook pad 98 can contact and attach to loop pad 99 and secure arm wrap band 94 to the arm. When arm wrap band 94 is wrapped around the arm of a person, as shown in FIG. 22, hex pad 96 can contact the person's arm wherein hexagonal compressible protrusions 12, when pressed against the person's arm, can induce a proprioceptive effect on exercise carried out by the person. In some embodiments, hex pad 96 can be sewn to band 94 whereas in other embodiments, hex pad 96 can releasably attached to band 94 using a hook and loop mechanism to allow hex pad 96 to be positioned anywhere along band 94 as desired

Referring to FIGS. 31 and 32, an alternate embodiment of arm wrap band 94 is shown. In this embodiment, arm wrap band 94 can comprise inflatable air bladder 101 disposed therein. Air bladder 101 can be inflated by pumping air in through inflation valve 93, as well known to those skilled in the art. In this embodiment, one end of arm wrap band 94 can be pulled through loop 97 to attach arm wrap band 94 to the person. In some embodiments, hook pad 98 can attach to loop pad 99 after passing through loop 97 thereby securing arm wrap band 94 to the arm. When air bladder 101 is inflated, the proprioceptive effect of hex pad 96 can be accentuated as the person exercises.

Referring to FIGS. 24 to 26, one embodiment of leg wrap band 100 is shown. In some embodiments, leg wrap band 100 can comprise of band 102 further comprising of hex pad 104 and hook pad 106 disposed on an inwardly facing surface thereof, and loop pad 108 disposed on an outwardly facing surface thereof. In some embodiments, leg wrap band 100 can be comprised of one or more of spandex, silicone, latex rubber, synthetic rubber, neoprene and any other functionally equivalent material as well known to those skilled in the art. In some embodiments, hex pad 104 can comprise a plurality of hexagonal compressible protrusions 12, separated by channels 14, disposed thereon. When leg wrap band 100 is wrapped around a leg of a person, either around the thigh or calf, hook pad 106 can contact and attach to loop pad 108 and secure leg wrap band 100 to the leg. When leg wrap band 100 is wrapped around the leg of a person, as shown in FIG. 26, hex pad 104 can contact the person's leg wherein hexagonal compressible protrusions 12, when pressed against the person's leg, can induce a proprioceptive effect on exercise carried out by the person. In some embodiments, hex pad 104 can be sewn to band 100 whereas in other embodiments, hex pad 104 can releasably attached to band 100 using a hook and loop mechanism to allow hex pad 104 to be positioned anywhere along band 100 as desired.

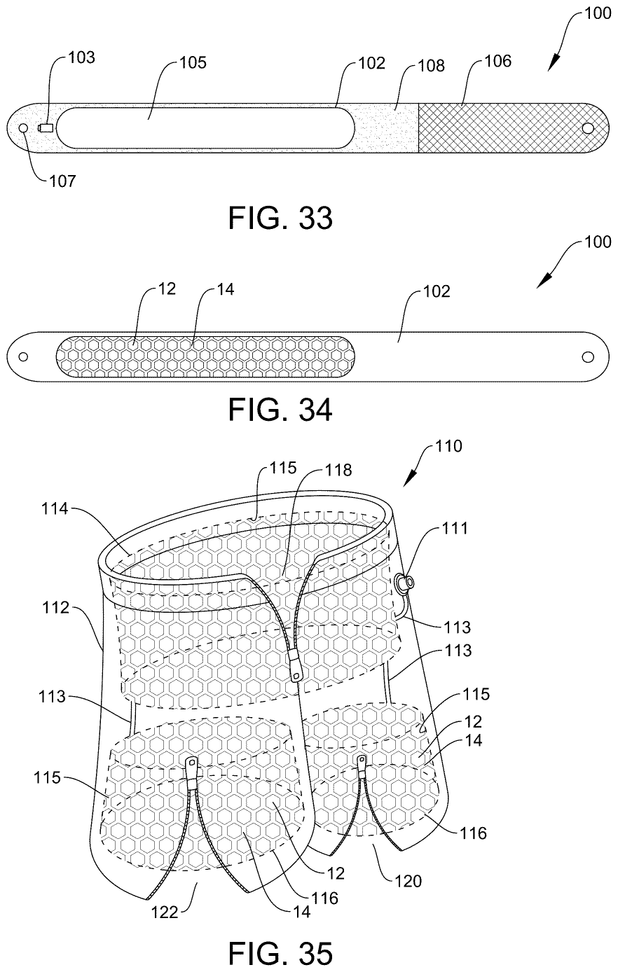

Referring to FIGS. 33 and 34, an alternate embodiment of leg wrap band 100 is shown. In this embodiment, leg wrap band 100 can comprise inflatable air bladder 105 disposed therein. Air bladder 105 can be inflated by pumping air in through inflation valve 103, as well known to those skilled in the art. In this embodiment, one end of leg wrap band 100 can be pulled through loop 107 to attach let wrap band 100 to the person. In some embodiments, hook pad 106 can attach to loop pad 108 after passing through loop 107 thereby securing leg wrap band 100 to the leg. When air bladder 105 is inflated, the proprioceptive effect of hex pad 104 can be accentuated as the person exercise.

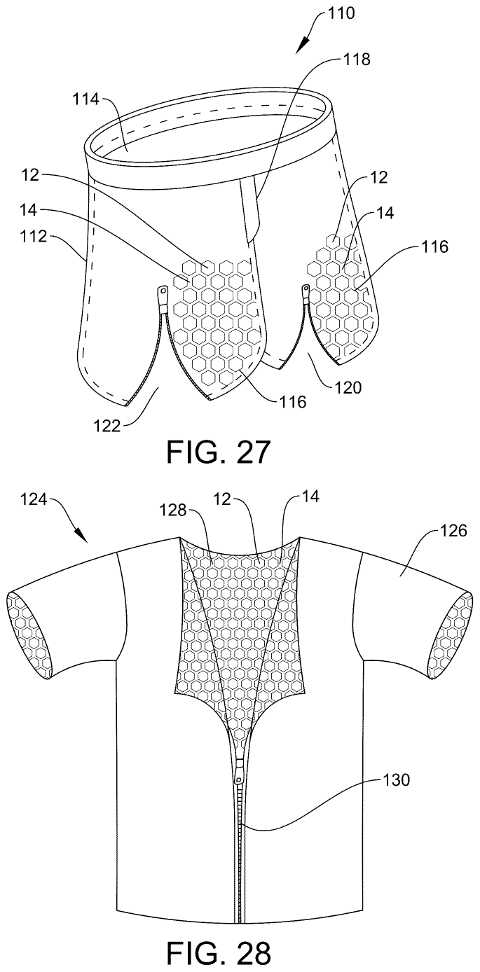

Referring to FIG. 27, one embodiment of exercise shorts 110 is shown. In some embodiments, exercise shorts 110 can comprise of outer shell 112, inner liner 114 and one or more hex pads 116 disposed on an inwardly facing surface thereof. In some embodiments, shorts 110 can comprise fly fastener 118 and can further comprise of one or both of inner seam fastener 120 and outer seam fastener 122 for securing exercise shorts 110 to a person. In some embodiments, fasteners 118, 120 and 122 can comprise of zippers or other functionally equivalent mechanism as well known to those skilled in the art. In some embodiments, exercise shorts 110 can be comprised of one or more of spandex, silicone, latex rubber, synthetic rubber, neoprene and any other functionally equivalent material as well known to those skilled in the art. In some embodiments, hex pads 116 can comprise a plurality of hexagonal compressible protrusions 12, separated by channels 14, disposed thereon. When exercise shorts 110 is worn by a person, hex pads 116 can contact the person's upper thighs, hips and posterior wherein hexagonal compressible protrusions 12, when pressed thereagainst, can induce a proprioceptive effect on exercise carried out by the person. In some embodiments, hex pad 116 can be sewn to shorts 110 whereas in other embodiments, hex pad 116 can releasably attached to shorts 110 using a hook and loop mechanism to allow hex pad 116 to be positioned anywhere within shorts 110 as desired.

Referring to FIG. 35, an alternate embodiment of exercise shorts 110 is shown. In this embodiment, exercise shorts 110 can comprise one or more inflatable air bladders 115 disposed therein. Air bladders 115 can be inflated by pumping air in through inflation valve 111, which can couple to air bladders 115 via air pipes 113, as well known to those skilled in the art. When air bladders 115 are inflated, the proprioceptive effect of hex pad 116 can be accentuated as the person exercises. In some embodiments, hex pad 116 can be sewn to shorts 110 whereas in other embodiments, hex pad 116 can releasably attached to shorts 110 using a hook and loop mechanism to allow hex pad 116 to be positioned anywhere within shorts 110 as desired.

Referring to FIG. 28, one embodiment of exercise shirt 124 is shown. In some embodiments, exercise shirts 124 can comprise outer shell 126 and inner liner 128. In some embodiments, exercise shirt 124 can comprise fastener 130 for securing exercise shirt 124 to a person. In some embodiments, fastener 130 can comprise of a zipper or other functionally equivalent mechanism as well known to those skilled in the art. In some embodiments, exercise shirt 124 can be comprised of one or more of spandex, silicone, latex rubber, synthetic rubber, neoprene and any other functionally equivalent material as well known to those skilled in the art. In some embodiments, inner liner 128 can comprise a plurality of hexagonal compressible protrusions 12, separated by channels 14, disposed thereon. When exercise shirt 124 is worn by a person, inner liner 128 can contact the person's upper body wherein hexagonal compressible protrusions 12, when pressed thereagainst, can induce a proprioceptive effect on exercise carried out by the person.

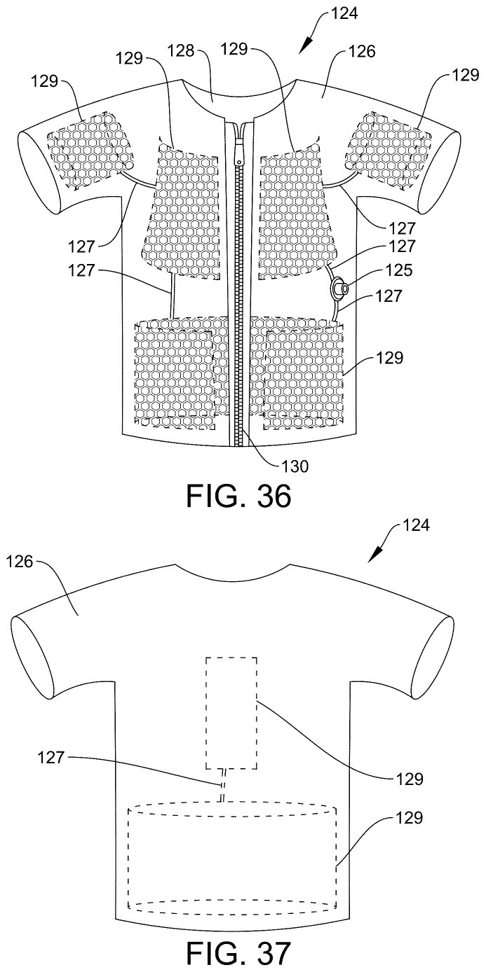

Referring to FIGS. 36 and 37, an alternate embodiment of exercise shirt 124 is shown. In this embodiment, exercise shirt 124 can comprise one or more inflatable air bladders 129 disposed therein. Air bladders 129 can be inflated by pumping air in through inflation valve 125, which can couple to air bladders 129 via air pipes 127, as well known to those skilled in the art. When air bladders 129 are inflated, the proprioceptive effect of hexagonal compressible protrusions 12 can be accentuated as the person exercises.

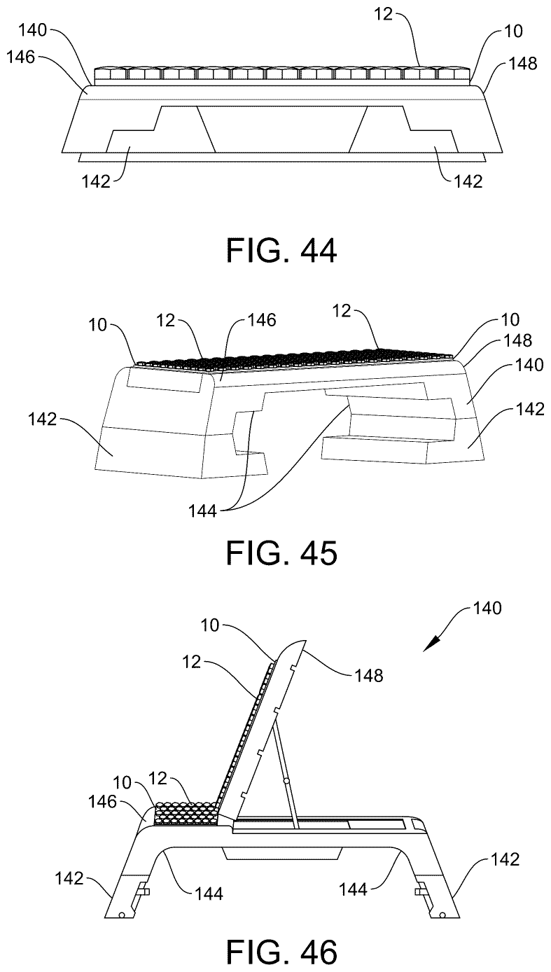

Referring to FIGS. 44 to 46, in some embodiments, a step up bench can comprise a mat of compressible protrusions disposed thereon. In the illustrated embodiments, step up bench 140 can comprise fold out legs 142 that can retract into and extend outward from recess 144. In some embodiments, step up bench 140 can comprise of seat portion 146 and back rest 148 that can pivot from a flat position, as shown in FIGS. 44 and 45, to an inclined position as shown in FIG. 46. In some embodiments, one or both of seat portion 146 and back rest 148 can comprise mat 10 disposed thereon, with mat 10 comprising a plurality of compressible protrusions 12 disposed thereon. In some embodiments, back rest 148 can be narrow relative to the width of a person's back so as to create an imbalance to urge the person to have their back in a correct position relative to back rest 148 during exercise. The compressible protrusions disposed on step up bench 140 can help boost fitness for full body workouts.

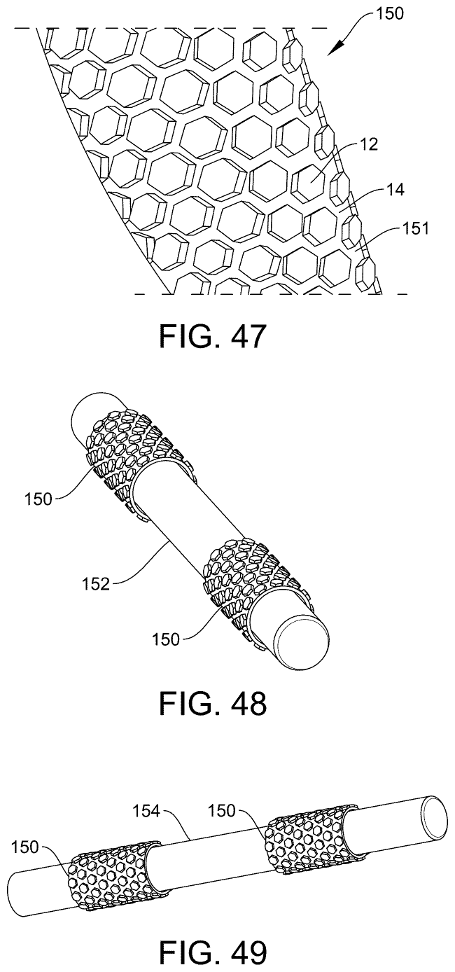

In some embodiments, a fat hand grip for a dumbbell handle or a bar for exercise equipment can be provided with a plurality of compressible protrusions disposed thereon, as shown in FIG. 47, where grip 150 can comprise of substrate 151 having a plurality of compressible protrusions 12 disposed on an outer surface thereof, the protrusions separated by channels 14. In some embodiments, grip 150 wraps around a bar increasing its diameter. The larger diameter can cause greater muscle activation in the arms of a person exercising with the bar wrapped with grip 150. In some embodiments, grip 150 can be wrapped around the hand grips of bar 152 that can be used with exercise machines as well known to those skilled in the art, as shown in FIG. 48. In some embodiments, grip 150 can be wrapped around the hand gripping portions of pull-up bar 154, as shown in FIG. 49.

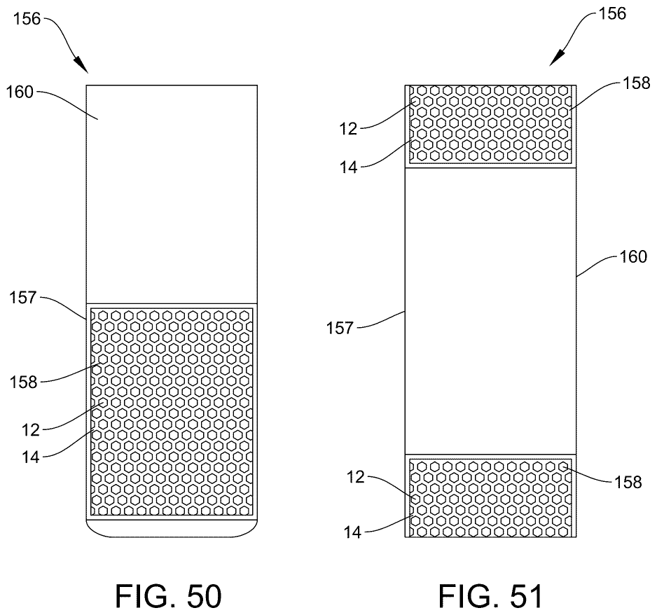

Referring to FIGS. 50 and 51, an embodiment of insert pad 156 is shown. In some embodiments, insert pad 156 can comprise of a silicone rubber or neoprene (or other like material) substrate 157 further comprising one or more protrusion pads 158 that can each comprise of a plurality of compressible protrusions 12 disposed thereon and channels 14 disposed therebetween. In some embodiments, insert pad 156 can comprise attachment pad 158 disposed for removably attaching insert pad 156 to a garment or belt represented by reference numeral 160. In some embodiments, attachment pad 158 can comprise of a hook and loop fastener mechanism, such as Velcro® to allow pad 156 to be releasably attached to garment or belt 160 and to be positioned thereon as desired. In some embodiments, insert pads 156 can be inserted into pockets disposed on garments. In further embodiments, the pockets can comprise openings disposed therethrough to align with and thereby allow protrusions 12 to pass through to allow for contact with the skin of the person wearing the garment. In some embodiments, insert pads 156 can be attached to strategic locations on a person, such as on: waist straps, bicep straps, calf straps, shoulder straps, quadricep straps, shirts, pants, tights and on any other type of clothing intended for athletic activity. The garments comprising insert pads 156 disposed thereon can be worn before, during or after athletic activity, and can also be worn as part of a regimen for post surgery recovery.

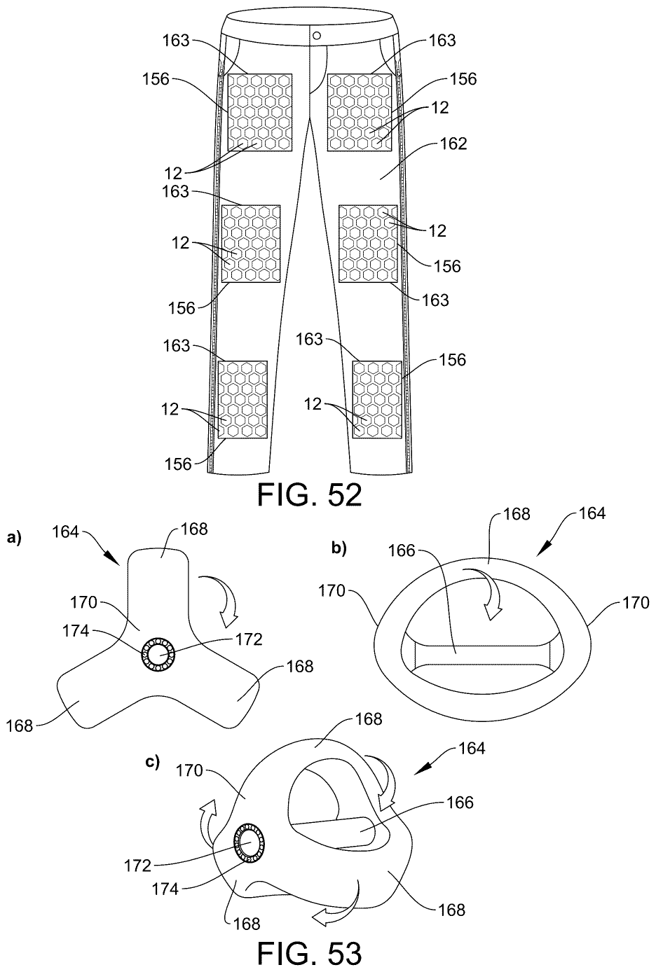

Referring to FIG. 52, an example of a garment comprising insert pads 156 is shown. In the illustrated embodiment, pant garment 162 can comprise one or more insert pads 156 disposed thereon, with each insert pad 156 disposed in a pocket 163 disposed on pant garment 162.

Referring to FIGS. 53a), b) and c), one embodiment of dumbbell 164 is shown. Dumbbell 164, as presented herein, is an improvement of a dumbbell disclosed in Applicant's earlier patent publication no. US2021/0236873A1, which is incorporated by reference into this application in its entirety. In some embodiments, dumbbell 164 can comprise a plurality of curved weight segments 168 disposed between a pair of end faces 170. The physical dimensions and materials used in weight segments 168 can be selected to provide dumbbells 164 having various masses or weights. In some embodiments, dumbbell 164 can further comprise substantially straight handle segment 166 disposed between end faces 170 along longitudinal axis 167, wherein handle segment 166 in between the plurality of weight segments 168. In some embodiments, handle segment 166 can comprise of opening 172 extending therethrough. In some embodiments, handle segment 166 can be rotatably attached to dumbbell 164 via bearings 174 disposed on each end face 170.

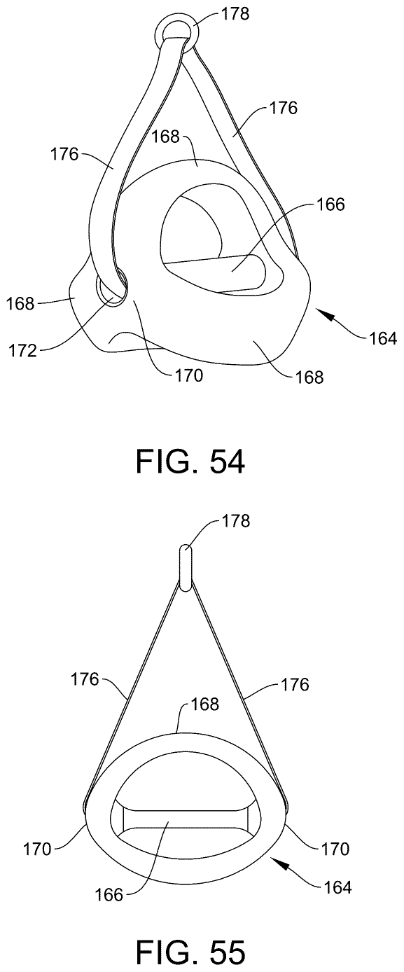

Referring to FIGS. 54 and 55, dumbbell 164 is shown having strap 176 placed through opening 172 to form a loop further comprising loop or ring 178 attached thereto so as to enable attaching ring 178 to weight resistance exercise machines, such as those using cables to pull on a weight stack as well known to those skilled in the art, so as to provide a weighted handle for use with such exercise machines.

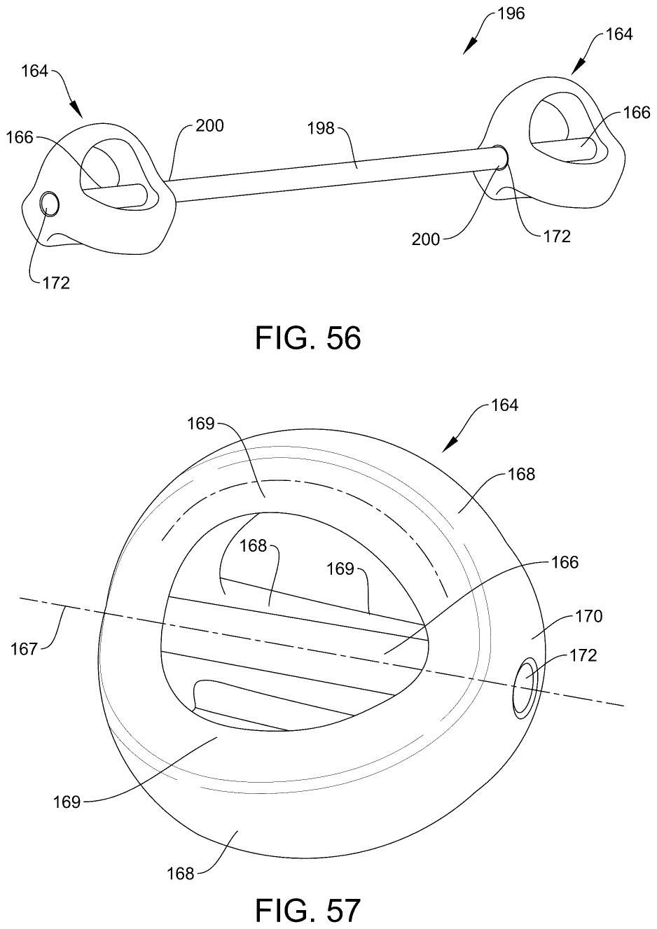

Referring to FIG. 56, one embodiment of barbell 196 is shown using dumbbells 164 as weights on opposing ends of bar 198. In some embodiments, the ends of bar 198 can be inserted into openings 172 of dumbbells 164 and can be held in place by releasable attachment mechanism 200. In some embodiments, releasable attachment mechanism 200 can comprise one or more of threading means, spring-loaded catch devices, spring-loaded ball catch devices, and any other releasable attachment mechanisms as well known to those skilled in the art.

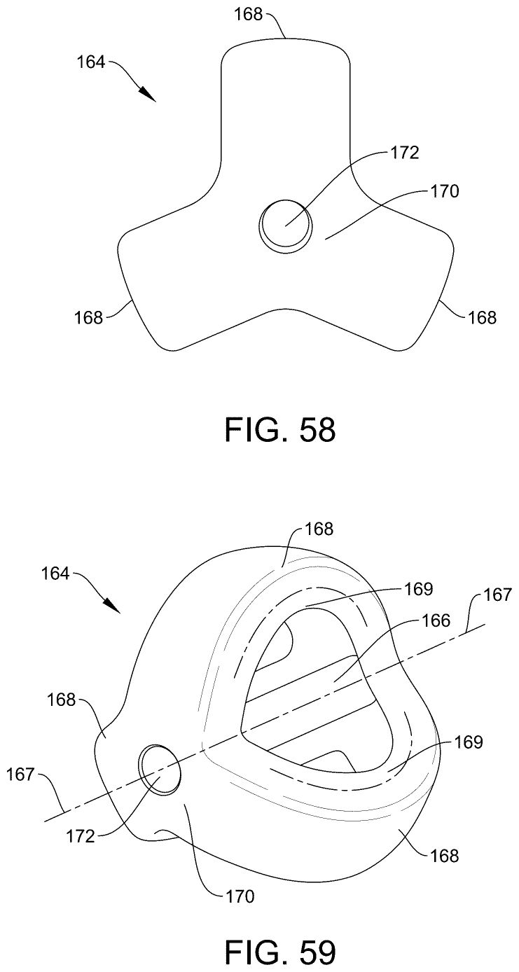

Referring to FIGS. 57, 58, and 59, one embodiment of dumbbell 164 is shown. In some embodiments, dumbbell 164 can comprise of handle 166 that can be substantially linearly straight along a longitudinal axis forming end faces 170 on both ends thereof. In some embodiments, dumbbell 164 can comprise one or more curved weight segments 168 following a curvilinear path between end faces 170. In the illustrated embodiment, dumbbell 164 is shown with three weight segments 168 spaced equidistant about handle 166 such that weight segments 168 are disposed approximately 120° apart from each other. The weight or mass of dumbbell 164 can be a function of the mass density of the material used to form dumbbell 164 and the cross-sectional dimensions of weight segments 168. In some embodiments, dumbbell 164 can be comprised metal, such as steel or cast iron. In other embodiments, dumbbell 164 can be comprised of rubber, such as high-density rubber as known to those skilled in the art. In further embodiments, dumbbell 164 can be comprised of metal, as noted above, covered in rubber or high-density rubber. In yet other embodiments, dumbbell 164 can be comprised of a shell of rubber or high-density plastic, such as high-density polyethylene (HDPE), or other functional equivalents as well known by those skilled in the art, that can be filled with metal beads, ball bearings, lead shot, sand or water, or other suitable media, as well known by those skilled in the art for producing exercise weight equipment, for producing a dumbbell of a desired mass or weight.

In some embodiments, one or more weight segments 168 can be curved in configuration to form arc 169 as shown in FIGS. 57 and 59. In some embodiments, arc 169 can provide additional space between weight segments 168 and handle 166 for a person to place their hand therearound while keeping the overall size of dumbbell 164 smaller than conventional dumbbells. In some embodiments, this embodiment of dumbbell can be comprised of metal, non-metal material with metal disposed therein or other suitable materials well known to those skilled in the art of making dumbbells. In some embodiments, the overall weight or mass of dumbbell 164 can be a function of the weight or mass of handle 166 and weight segments 168, which can further be a function of its thickness, its cross-sectional area and the mass density of the material used to form dumbbell 164.

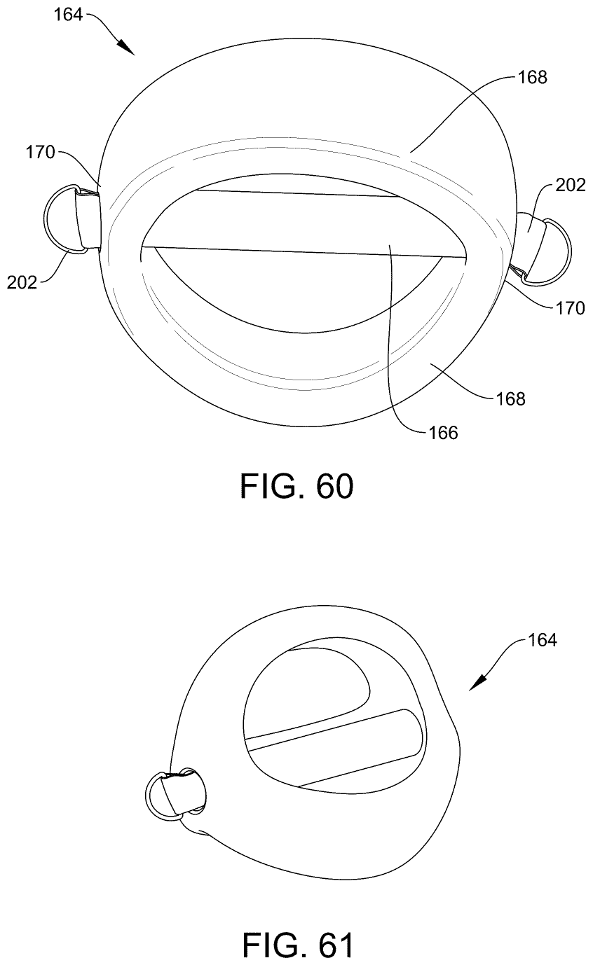

Referring to FIG. 60, another embodiment of dumbbell 164 is shown. In this illustrated embodiment, dumbbell 164 can comprise of loop or ring 202 disposed on one or both of end faces 170. Referring to FIG. 61, illustrated is a way of exercising with dumbbell 164 by lifting and moving dumbbell 164 similar to how one would exercise with a kettle bell as well known to those skilled in the art.

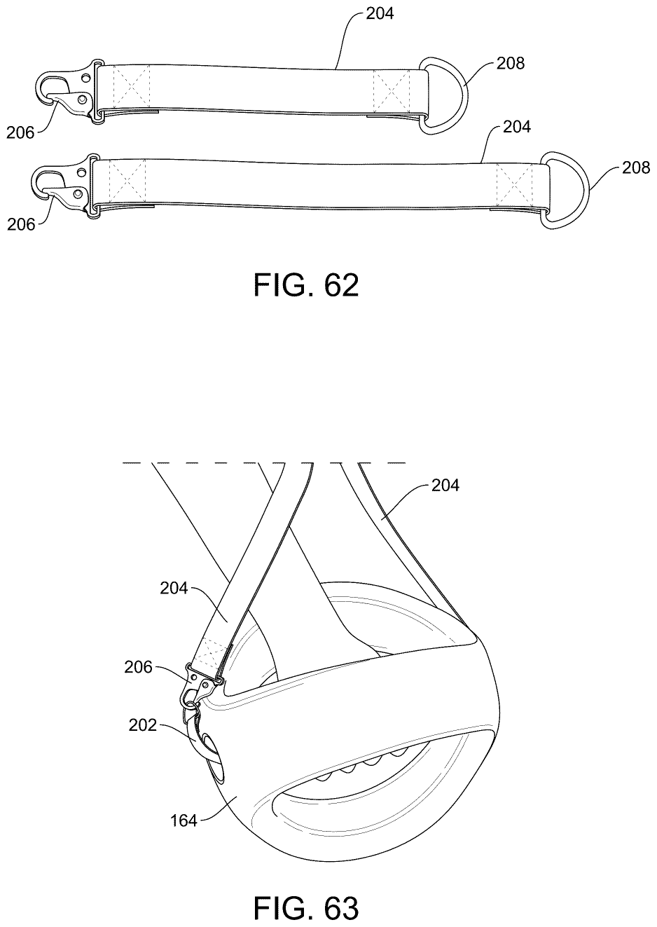

Referring to FIG. 62, embodiments of straps 204 for use with dumbbell 164 are shown, which can comprise of clip 206 disposed on one end thereof and loop or ring 208 on an opposing end thereof. In some embodiments, clip 206 attach to loop or ring 202 of dumbbell 164 wherein loop or ring 208 of strap 204 can attach to an exercise machine, as described in more detail below. Referring to FIG. 63, one embodiment of dumbbell 164 is shown with two straps 204 attached thereto for attachment to an exercise machine (not shown) such that dumbbell 164 can function as the handle coupled to the exercise machine wherein the additional weight or mass of dumbbell 164 provides additional resistance to enhance the effects of exercise on the exercise machine.

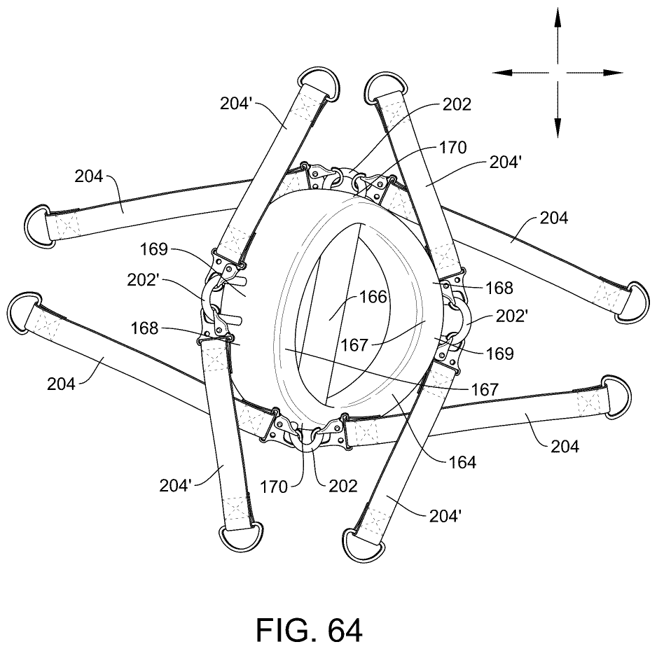

Referring to FIG. 64, another embodiment of dumbbell 164 is shown. In this embodiment, dumbbell 164 can comprise of loops or rings 202′ disposed on apex 169 of arc 169 on one or more of weight segments 168 in addition to loops or rings 202 disposed on end faces 170. In some embodiments, one or more straps 204 can be attached to one or more loops or rings 202 and/or one or more straps 204′ can be attached to one or more loops or rings 202′. Thus, in some embodiments, dumbbell 164 can be attached to an exercise machine in a variety of attachment orientations with the exercise machine such that dumbbell 164 can function as the handle coupled to the exercise machine wherein the additional weight or mass of dumbbell 164 provides additional resistance to enhance the effects of exercise on the exercise machines.

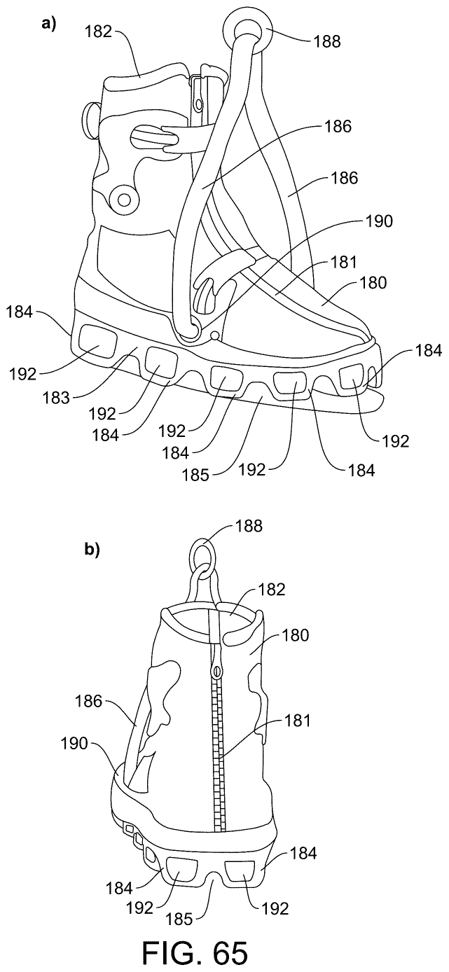

Referring to FIGS. 65a) and b), an embodiment of a galosh for wearing on footwear during physical exercise is illustrated. In some embodiments, galosh 180 can be placed onto a piece of footwear 182 and secured thereto with zipper 181. In some embodiments, galosh 180 can comprise of sole 183 having a plurality of compressible protrusions 184 extending downwardly therefrom with channels 185 disposed therebetween. In some embodiments, one or more of protrusions 184 can comprise of opening or void 192 disposed therein. In some embodiments, galosh 180 can further comprise of strap 186 operatively coupled thereto via attachment point 190 disposed on either side of galosh 180. Strap 186 can be formed into a loop further comprising loop or ring 188 attached thereto so as to enable attaching ring 188 to weight resistance exercise machines, such as those using cables to pull on a weight stack as well known to those skilled in the art, so as to provide a weighted handle for use with such exercise machines.

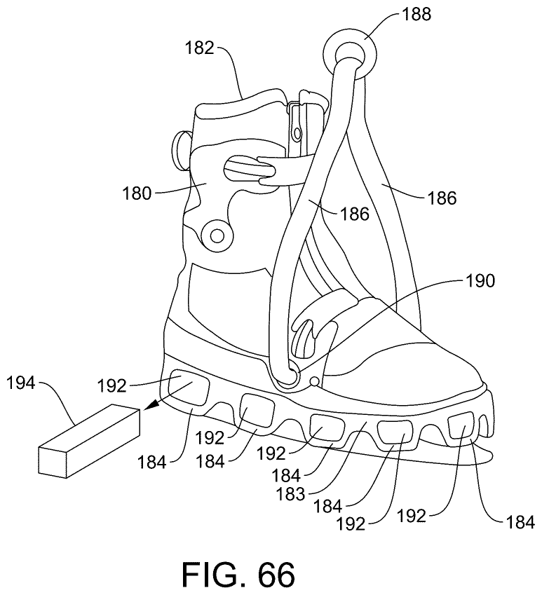

Referring to FIG. 66, another embodiment of galosh 180 is illustrated wherein one or more weights 194 can be placed into openings 192 to provide further weight or resistance to the combination of galosh 180 on footwear 182 when worn by a person during physical exercise when ring 188 is attached to a weight resistance machine.

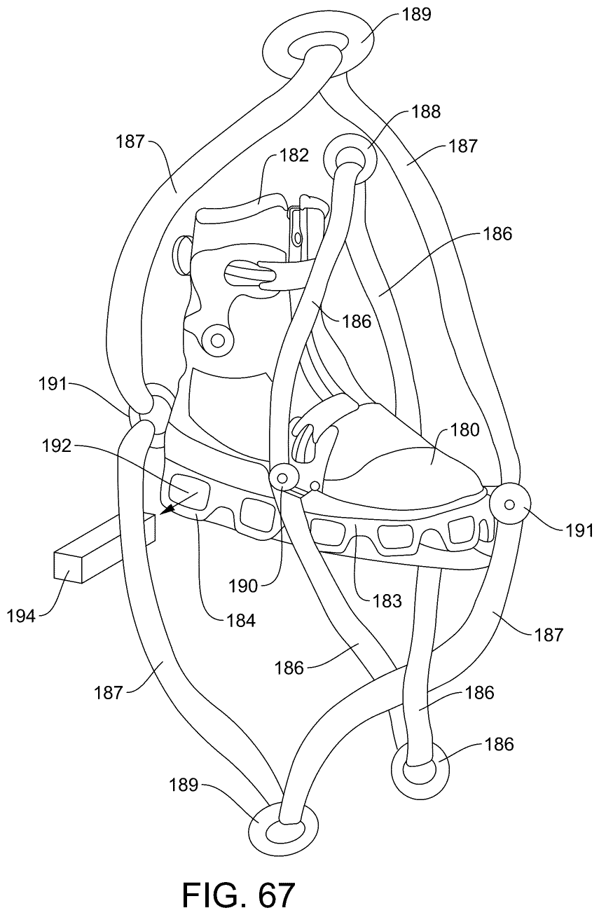

Referring to FIG. 67, another embodiment of galosh 180 for wearing on footwear during physical exercise is illustrated. In some embodiments, galosh 180 can be placed onto a piece of footwear 182 and secured thereto with zipper 181. In some embodiments, galosh 180 can comprise of sole 183 having a plurality of compressible protrusions 184 extending downwardly therefrom with channels 185 disposed therebetween. In some embodiments, one or more of protrusions 184 can comprise of opening or void 192 disposed therein. In some embodiments, galosh 180 can further comprise of strap 186 operatively coupled thereto via attachment point 190 disposed on either side of galosh 180. Strap 186 can be formed into a loop further comprising loop or ring 188 attached thereto so as to enable attaching ring 188 to weight resistance exercise machines, such as those using cables to pull on a weight stack as well known to those skilled in the art, so as to provide a weighted handle for use with such exercise machines. In some embodiments, galosh 180 can further comprise strap 187 operatively coupled attached thereto via attachment point 191 on the toe and heel of galosh 180. Strap 187 can be formed into a loop further comprising loop or ring 189 attached thereto so as to enable attaching ring 189 to weight resistance exercise machines, such as those using cables to pull on a weight stack as well known to those skilled in the art, so as to provide a weighted handle for use with such exercise machines. In some embodiments, galosh 180 as illustrated can comprise one or more weights 194 can be placed into openings 192 to provide further weight or resistance to the combination of galosh 180 on footwear 182 when worn by a person during physical exercise when one or both of rings 188 and 189 are attached to a weight resistance machine.

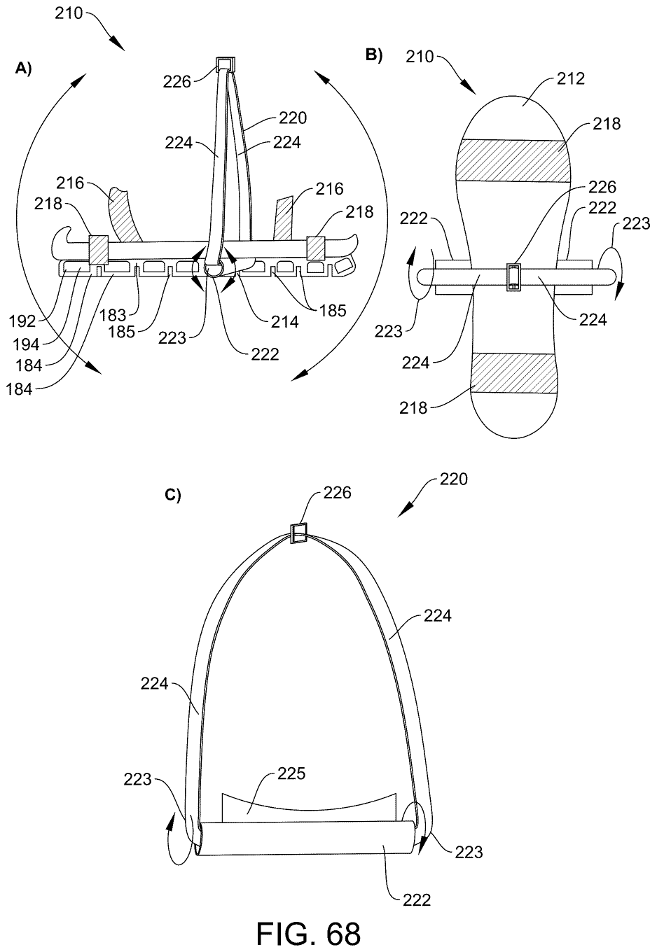

Referring to FIG. 68a) to c), one embodiment of foot attachment device 210 for use with a weight resistance machine is shown. In some embodiments, device 210 can comprise of footboard 212 having straps 216 and 218 disposed thereon for attaching device 210 to a foot or footwear of a person. In some embodiments, device 210 can further comprise of sole 183 having a plurality of compressible protrusions 184 extending downwardly therefrom with channels 185 disposed therebetween. In some embodiments, one or more of protrusions 184 can comprise of opening or void 192 disposed therein. In some embodiments, device 210 can further comprise of strap 224 operatively coupled to bar 222 via pivot attachment 223 disposed on either side of bar 222, wherein bar 222 can be disposed in opening 214 disposed in sole 183. In this illustrated embodiment, opening 214 can be disposed traversing across the width of footboard 212. In some embodiments, strap 224 can be formed into a loop further comprising loop or ring 226 attached thereto so as to enable attaching ring 226 to weight resistance exercise machines, such as those using cables to pull on a weight stack as well known to those skilled in the art, so as to provide a weighted handle for use with such exercise machines. In some embodiments, bar 222 can further comprise of ridge 225 configured for attaching bar 222 to sole 183 when bar 222 is placed in opening 214. In some embodiments, device 210 can further comprise one or more weights 194 can be placed into openings 192 to provide further weight or resistance to device 210 when worn by a person during physical exercise when one ring 226 is attached to a weight resistance machine.

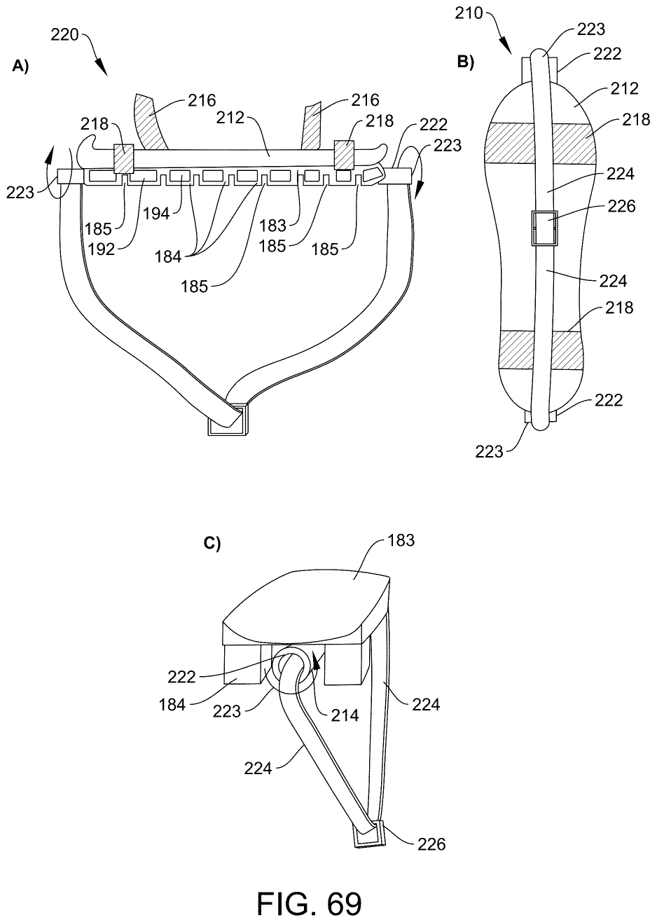

Referring to FIG. 69a) to c), another embodiment of device 210 is shown, which differs from the embodiment shown in FIG. 68a) to c) by bar 222 disposed in opening 214 disposed lengthwise in sole 183 from the toe to the heel portions of footboard 212, as opposed to side-to-side thereof.

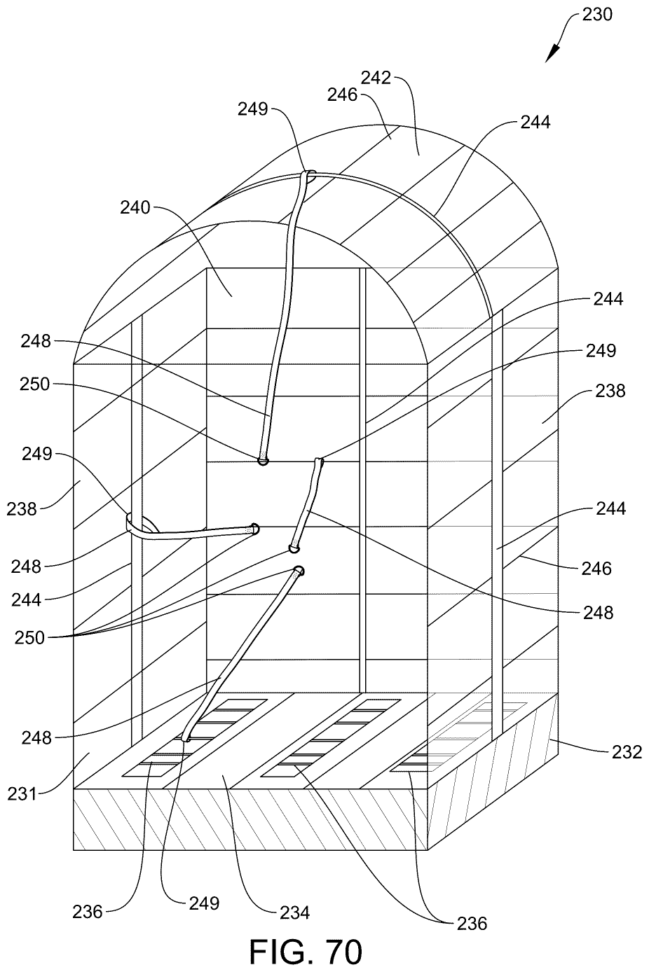

Referring to FIG. 70, one embodiment of cage 230 for use with dumbbell 164, galosh 180, and device 210 is shown. In some embodiments, cage 230 can comprise of elevated base 232, sidewalls 238, rear wall 240, and roof 242 supported thereon to form opening 231 to allow a person to enter cage 230. In some embodiments, base 232 can comprise of one or more floor portions 234 and one or more grate portions 236 for attaching resistance band 248 thereto at attachment point 249. In some embodiments, one or more of sidewalls 238, rear wall 240, and roof 240 can comprise of first attachment members 244 disposed therein, and of one or more second attachment members 246 disposed therein wherein first and second attachment members are substantially orthogonal to each other. In some embodiments, one or both of first and second attachment members can be configured for attaching resistance bands 248 thereto at attachment points 249. In some embodiments, one or more of resistance bands 248 can comprise attachment loop or clip 250 disposed on the loose end thereof. In some embodiments, one of more of dumbbell 164, galosh 180, and device 210 can be attached to resistance bands 248 via attachment loop or clip 250 wherein a person using dumbbell 164 or wearing galosh 180 or device 210 can perform exercise movements in cage 230 with resistance bands 248 with the added resistance of the weight of dumbbell 164 and/or the inclusion of added weight 194 to sole 183 of galosh 180 or device 210.

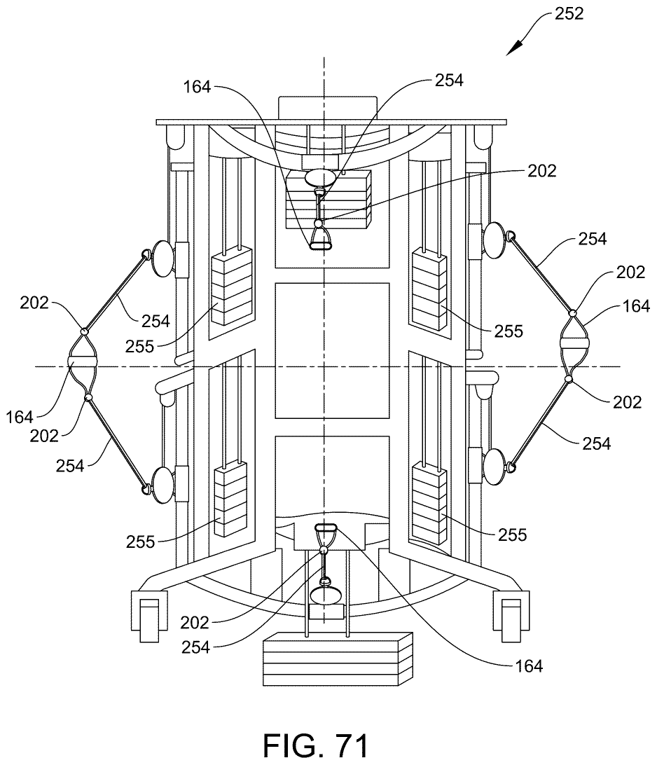

Referring to FIG. 71, one embodiment of cable exercise machine 252 for use with dumbbell 164, galosh 180, and device 210 is shown. In some embodiments, cable exercise machine 252 can comprise of one or more cables 254 operatively coupled to weight stacks 255. In some embodiments, one or more of cables 254 can attach to one or more of dumbbell 164, galosh 180, and device 210. In the illustrated embodiment, cables 254 can be attached to dumbbell 164 via loop or ring 202 disposed thereon. Similarly, in some embodiments, galosh 180 can be attached to cables 254 via its loops 188 or 189. Similarly, in some embodiments, device 210 can be attached to cables 254 via loop or ring 226. In some embodiments, one of more of dumbbell 164, galosh 180, and device 210 can be attached to cables 254 wherein a person using dumbbell 164 or wearing galosh 180 or device 210 can perform exercise movements in cable exercise machine 252 with the added resistance of the weight of dumbbell 164, and/or the inclusion of added weight 194 to sole 183 of galosh 180 or device 210, in combination with weight stacks 255.

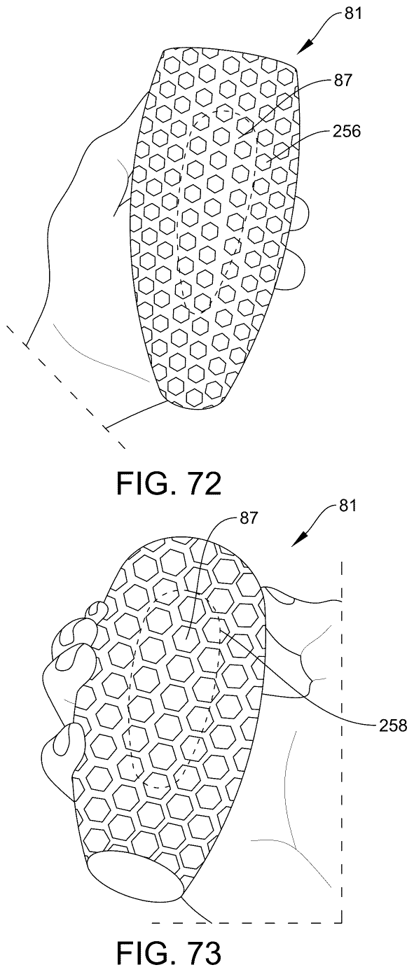

Referring to FIG. 72, an alternate embodiment of hand weight 81, as shown in FIGS. 14 and 15, are illustrated. In some embodiments, hand weight 81 can comprise a plurality of hemispherical compressible protrusions 256 extending outwardly from an outer surface thereof.

Referring to FIG. 73, a further embodiment of hand weight 81 is illustrated. In some embodiments, hand weight 81 can comprise a plurality of hexagonal compressible protrusions 258 extending outwardly from an outer surface thereof.

In some embodiments, hand weight 81 can comprise a conical or cone shape that can allow for a more comfortable or complete grip therearound when in the hand of a person gripping it. Holding or gripping hand weight 81 can provide additional resistance to a person's movement when performing physical exercise thereby enhancing the effects thereof.

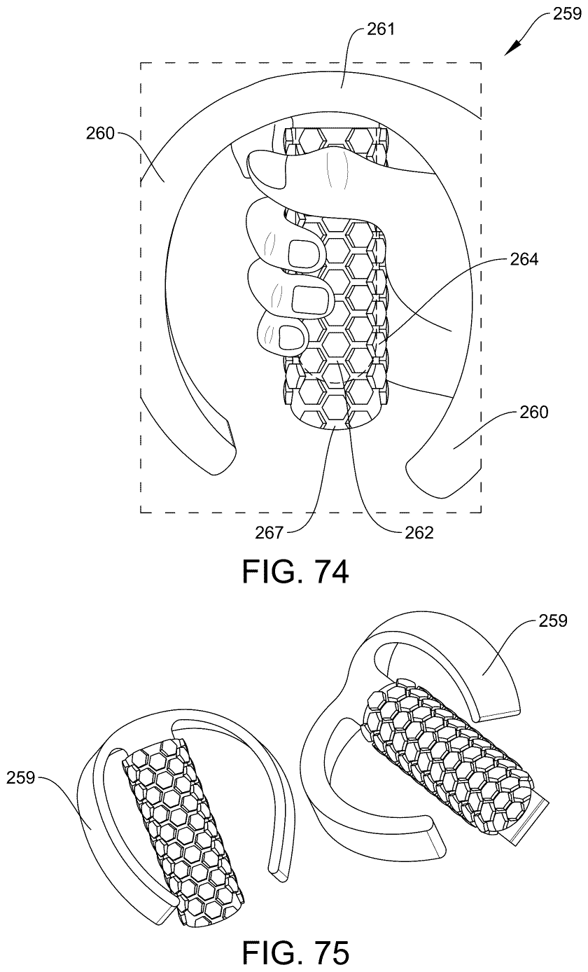

Referring to FIGS. 74 and 75, one embodiment of dumbbell 259 is shown wherein dumbbell 259 can comprise of cylindrical handle 262 having end face 261 disposed at one end thereof, further comprising a plurality of arcuate weight segments 260 extending from end face 261.

In some embodiments, arcuate weight segments 260 can provide additional space between weight segments 260 and handle 262 for a person to place their hand therearound while keeping the overall size of dumbbell 259 smaller than conventional dumbbells. In some embodiments, this embodiment of dumbbell can be comprised of metal, non-metal material with metal disposed therein or other suitable materials well known to those skilled in the art of making dumbbells. In some embodiments, the overall weight or mass of dumbbell 259 can be a function of the weight or mass of handle 262 and weight segments 260, which can further be a function of its thickness, its cross-sectional area, and its mass density. In some embodiments, dumbbell 259 can further comprise grip 264 disposed around handle 262 wherein grip 264 can comprise a plurality of compressible protrusions 267 extending outwardly from an outer surface thereof.

Referring to FIG. 75, illustrated is a person using a pair of dumbbells 259 during boxing training exercises, wherein the person is using dumbbells 259 in substitution of boxing gloves to provide additional resistance during the boxing training exercises.

In some embodiments, compressible protrusions 256, 258, and 267 can comprise one or more of a group comprising rubber, thermoplastic rubber, foam rubber, polyurethane rubber, silicone rubber, thermoplastic resin and thermoplastic elastomer, wherein the compressible protrusions aid in inducing a proprioceptive effect to a person's CNS during exercise.

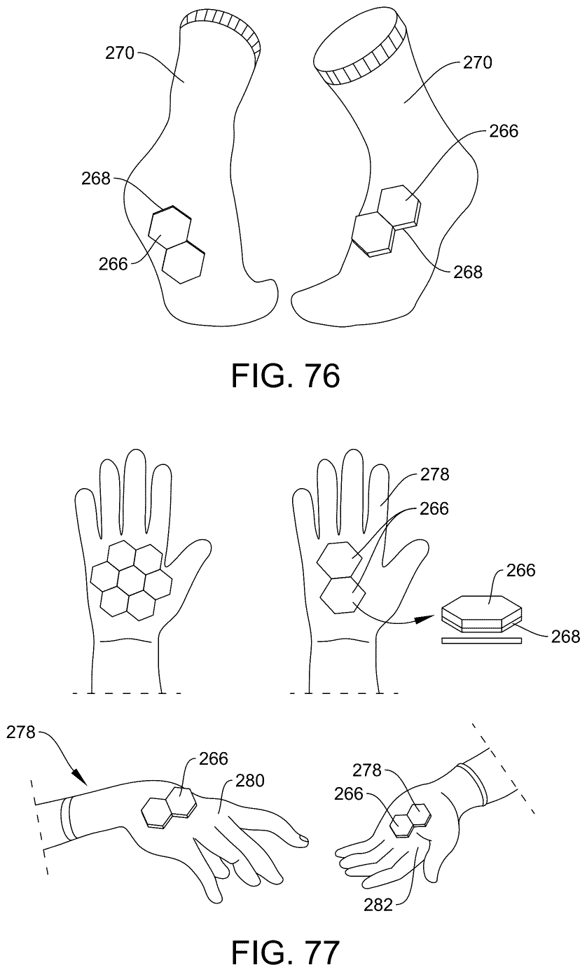

Referring to FIG. 76, one embodiment of a weight for attaching to clothing is illustrated. In some embodiments, weight member 266 can be attached to article of clothing 270. In some embodiment, weight member 266 can be attached to the clothing using a layer of hook and loop fastener 268 that can attach to the fabric of the clothing. In some embodiments, the hook and loop fastener can comprise a Velcro® fastener. In the illustrated embodiment, article of clothing 270 is a sock although weight member 266 can be applied to any article of clothing that can attach to hook and loop fastener 268. When weight member 266 is attached to an article of clothing, it can provide additional resistance to a person's movement when performing physical exercise thereby enhancing the effects thereof.

Referring to FIG. 77, one embodiment of a weight for attaching to clothing is illustrated. In some embodiments, weight member 266 can be attached to article of clothing such as glove 278. In some embodiment, weight member 266 can be attached to glove 278 a layer of hook and loop fastener 268 that can attach to the fabric of the clothing. In some embodiments, the hook and loop fastener can comprise a Velcro® fastener. In some embodiments, weight 266 can attach to dorsal side 280 of glove 278 whereas in other embodiments, weight 266 can attach to palm side 282 of glove 278. When weight member 266 is attached to glove 278, it can provide additional resistance to a person's movement when performing physical exercise thereby enhancing the effects thereof.

In some embodiments, weight 266 can be comprised of metal or be filled with sand, or other similar materials as well known to those skilled in the art, to provide weight or mass to weight 266.

Referring to FIGS. 84 and 85, one embodiment of a compressible protrusion for use on an insole for a shoe. In some embodiments, one or more of protrusions 314 and 316 can be attached to insole 312 using a hook and loop fastening mechanism. In some embodiments protrusions 314 can comprise a low density to compress to provide the proprioceptive effect. In some embodiments, protrusions 316 can comprise a high density to provide the acupressure effect. In some embodiments, protrusions 314 and 316 can comprise hook layer 320 disposed on a lower surface thereof, which can couple to loop surfaces 318 disposed on a top surface of insole 312. Hook layer 320 on protrusions 314 and 316 can allow these protrusions to be placed on any location on top of loop surfaces 318 so as to let a person add a proprioceptive effect or acupressure effect at any desired location on insole 312 once placed in a shoe worn by the person during physical exercise.

Although a few embodiments have been shown and described, it will be appreciated by those skilled in the art that various changes and modifications can be made to these embodiments without changing or departing from their scope, intent or functionality. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

1.-34. (canceled)

35. A garment to be worn by a person during exercise, comprising:
a) a pair of shorts configured to be worn by the person about their waist, hips and thighs, the pair of shorts comprising an outer shell;
b) an inner liner disposed within the outer shell, the inner liner comprising a plurality of compressible protrusions extending inwardly therefrom towards the person when the pair of shorts are worn by the person; and
c) a fastening mechanism configured to secure the pair of shorts to the person.

36. The garment as set forth in claim 35, wherein each of the plurality of compressible protrusions is substantially hexagonal in configuration.

37. The garment as set forth in claim 35, wherein each of the plurality of compressible protrusions comprises one or more of a group comprising rubber, thermoplastic rubber, foam rubber, polyurethane rubber, silicone rubber, thermoplastic resin and thermoplastic elastomer.

38. The garment as set forth in claim 35, wherein the fastening mechanism comprises one or more of a fly zipper, a waistband fastener, an outer seam zipper and an inner seam zipper.

39. The garment as set forth in claim 35, further comprising at least one inflatable bladder disposed therein.

40. A garment to be worn by a person during exercise, comprising:
a) a shirt configured to be worn by the person on their torso, the shirt comprising an outer shell;
b) an inner liner disposed within the outer shell, the inner liner comprising a plurality of compressible protrusions extending inwardly therefrom towards the person when the shirt is worn by the person; and
c) a fastening mechanism configured to secure the shirt to the person.

41. The garment as set forth in claim 40, wherein each of the plurality of compressible protrusions is substantially hexagonal in configuration.

42. The garment as set forth in claim 40, wherein each of the plurality of compressible protrusions comprises one or more of a group comprising rubber, thermoplastic rubber, foam rubber, polyurethane rubber, silicone rubber, thermoplastic resin and thermoplastic elastomer.

43. The garment as set forth in claim 40, wherein the fastening mechanism comprises one or more of a zipper, one or more buttons, one or more clasps and one or more snap fasteners.

44. The garment as set forth in claim 40, further comprising at least one inflatable bladder disposed therein.

45.-80. (canceled)

81. An exercise band for placing around an abdomen or limb of a person, comprising:
a) a band member comprising first and second ends, the band member further comprising an outward-facing surface and an inward-facing surface, the inward-facing surface configured to contact the abdomen or the limb of the person when the band member is placed therearound;
b) a plurality of compressible protrusions extending from the inward-facing surface; and
c) a fastening mechanism configured for securing the exercise band to the person.

82. The exercise band as set forth in claim 81, wherein each of the plurality of compressible protrusions is substantially hexagonal in configuration.

83. The exercise band as set forth in claim 81, wherein each of the plurality of compressible protrusions comprises one or more of a group comprising rubber, thermoplastic rubber, foam rubber, polyurethane rubber, silicone rubber, thermoplastic resin and thermoplastic elastomer.

84. The exercise band as set forth in claim 81, wherein the fastening mechanism comprises a hook and loop fastener.

85. The exercise band as set forth in claim 81, further comprising an inflatable bladder disposed therein.

86. The exercise band as set forth in claim 81, wherein the limb comprises an arm or a leg of the person.
